SEQUENCE LISTING (16 sequences)

SEQ ID NO: 1 - DNA, 37 bp, Artificial Sequence
  IF-RabM-S3.c
aaatttgtcgggcccatgaactttctacgtaagatag

SEQ ID NO: 2 - DNA, 39 bp, Artificial Sequence
  IF-RabM-S1-4.r
actaaagaaaataggcctttattctagaagcagagagga

SEQ ID NO: 3 - DNA, 609 bp, Artificial Sequence
  Synthesized M protein coding sequence
atgaactttctacgtaagatagtgaaaaattgcagggacgaggacactcaaaaaccctctcccgtgtcagcccctctggatgacgatgacttgtggcttccaccccctgaatacgtcccgctgaaagaacttacaagcaagaagaacatgaggaacttttgtatcaacggaggggttaaagtgtgtagcccgaatggttactcgttcaggatcctgcggcacattctgaaatcattcgacgagatatattctgggaatcataggatgatcgggttagtcaaagtagttattggactggctttgtcaggatctccagtccctgagggcatgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagaatctggtgtatcaacatgaacccgagagcatgtcaactatggtctgacatgtctcttcagacacaaaggtccgaagaggacaaagattcctctctgcttctagaataa

SEQ ID NO: 4 - DNA, 4903 bp, Artificial Sequence
  Construct number 1191
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccgcggatggcgaaaaacgttgcgattttcggcttattgttttctcttcttgtgttggttccttctcagatcttcgcctgcaggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 5 - DNA, 2349 bp, Artificial Sequence
  Expression cassette number 1066
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatgaactttctacgtaagatagtgaaaaattgcagggacgaggacactcaaaaaccctctcccgtgtcagcccctctggatgacgatgacttgtggcttccaccccctgaatacgtcccgctgaaagaacttacaagcaagaagaacatgaggaacttttgtatcaacggaggggttaaagtgtgtagcccgaatggttactcgttcaggatcctgcggcacattctgaaatcattcgacgagatatattctgggaatcataggatgatcgggttagtcaaagtagttattggactggctttgtcaggatctccagtccctgagggcatgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagaatctggtgtatcaacatgaacccgagagcatgtcaactatggtctgacatgtctcttcagacacaaaggtccgaagaggacaaagattcctctctgcttctagaataaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 6 - protein, 202 aa, Rabies virus
MNFLRKIVKNCRDEDTQKPSPVSAPLDDDDLWLPPPEYVPLKELTSKKNMRNFCINGGVKVCSPNGYSFRILRHILKSFDEIYSGNHRMIGLVKVVIGLALSGSPVPEGMNWVYKLRRTFIFQWADSRGPLEGEELEYSQEITWDDDTEFVGLQIRVIAKQCHIQGRIWCINMNPRACQLWSDMSLQTQRSEEDKDSSLLLE

SEQ ID NO: 7 - DNA, 6789 bp, Artificial Sequence
  Construct No. 1193
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccgcggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 8 - DNA, 4379 bp, Artificial Sequence
  Expression cassette number 1086
ctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatgaactttctacgtaagatagtgaaaaattgcagggacgaggacactcaaaaaccctctcccgtgtcagcccctctggatgacgatgacttgtggcttccaccccctgaatacgtcccgctgaaagaacttacaagcaagaagaacatgaggaacttttgtatcaacggaggggttaaagtgtgtagcccgaatggttactcgttcaggatcctgcggcacattctgaaatcattcgacgagatatattctgggaatcataggatgatcgggttagtcaaagtagttattggactggctttgtcaggatctccagtccctgagggcatgaactgggtatacaaattgaggagaacctttatcttccagtgggctgattccaggggccctcttgaaggggaggagttggaatactctcaggagatcacttgggatgatgatactgagttcgtcggattgcaaataagagtgattgcaaaacagtgtcatatccagggcagaatctggtgtatcaacatgaacccgagagcatgtcaactatggtctgacatgtctcttcagacacaaaggtccgaagaggacaaagattcctctctgcttctagaataaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtac

SEQ ID NO: 9 - DNA, 42 bp, Artificial Sequence
  IF-RabG-S2+4.c
tctcagatcttcgccaaattccctatttacacgataccagac

SEQ ID NO: 10 - DNA, 48 bp, Artificial Sequence
  IF-RabG-S1-4.r
actaaagaaaataggccttcacagtctggtctcacccccactcttgtg

SEQ ID NO: 11 - DNA, 1575 bp, Artificial Sequence
  Synthesized Rabies G protein coding sequence
atggttcctcaggctctcctgtttgtaccccttctggtttttccattgtgttttgggaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtga

SEQ ID NO: 12 - DNA, 4897 bp, Artificial Sequence
  Construct number 1192
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgccgcggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagcgatcgctcaccatcaccatcaccatcaccatcaccattaaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 13 - DNA, 3330 bp, Artificial Sequence
  Expression cassette No. 1071
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgccaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 14 - protein, 529 aa, Rabies virus
MAKNVAIFGLLFSLLVLVPSQIFAKFPIYTIPDKLGPWSPIDIHHLSCPNNLVVEDEGCTNLSGFSYMELKVGYILAIKMNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPDQLVNLHDFRSDEIEHLVVEELVRKREECLDALESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 15 - DNA, 6861 bp, Artificial Sequence
  Construct No. 1194
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacacgcgtggcgcgccctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgccgcggctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtacggccggccactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 16 - DNA, 5360 bp, Artificial Sequence
  Expression cassette number 1091
ctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgataaagcgtatattgtatcaggtatttccgtcggatacgaattattcgtacaagcttcttaagccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaatttgtcgggcccatggcgaaaaacgttgcgattttcggcttattgttttctcttcttgtgttggttccttctcagatcttcgccaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgcactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtgaaggcctattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcggggtaccgagctcgaattccgagtgtacttcaagtcagttggaaatcaataaaatgattattttatgaatatatttcattgtgcaagtagatagaaattacatatgttacataacacacgaaataaacaaaaaaacacaatccaaaacaaacaccccaaacaaaataacactatatatatcctcgtatgaggagaggcacgttcagtgactcgacgattcccgagcaaaaaaagtctccccgtcacacatatagtgggtgacgcaattatcttcaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaagtcgatggtcccgttccagtagttgtgtcgcccgagacttctagcccaggtggtctttccggtacgagttggtccgcagatgtagaggctggggtgtctgaccccagtccttccctcatcctggttagatcggccatccactcaaggtcagattgtgcttgatcgtaggagacaggatgtatgaaagtgtaggcatcgatgcttacatgatataggtgcgtctctctccagttgtgcagatcttcgtggcagcggagatctgattctgtgaagggcgacacgtactgctcaggttgtggaggaaataatttgttggctgaatattccagccattgaagctttgttgcccattcatgagggaattcttctttgatcatgtcaagatactcctccttagacgttgcagtctggataatagttcgccatcgtgcgtcagatttgcgaggagagaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggataagagcatgatagtgggtagtgccatcttgatgaagctcagaagcaacaccaaggaagaaaataagaaaaggtgtgagtttctcccagagaaactggaataaatcatctctttgagatgagcacttggggtaggtaaggaaaacatatttagattggagtctgaagttcttgctagcagaaggcatgttgttgtgactccgaggggttgcctcaaactctatcttataaccggcgtggaggcatggaggcaagggcattttggtaatttaagtagttagtggaaaatgacgtcatttacttaaagacgaagtcttgcgacaaggggggcccacgccgaattttaatattaccggcgtggccccaccttatcgcgagtgctttagcacgagcggtccagatttaaagtagaaaagttcccgcccactagggttaaaggtgttcacactataaaagcatatacgatgtgatggtatttgatggagcgtatattgtatcaggtatttccgtcggatacgaattattcgtac

(19) United States
(12) Patent Application Publication
(10) Pub. No.: US 2014/0227322 A1
(43) Pub. Date: Aug. 14, 2014

(54) RABIES VIRUS LIKE PARTICLE PRODUCTION IN PLANTS

(71) Applicants: Marc-Andre D'Aoust, Quebec (CA); Pierre-Olivier Lavoie, Quebec (CA); Louis-Philippe Vezina, Neuville (CA); Manon Couture, St. Augustin de Desmaures (CA)

(72) Inventors: Marc-Andre D'Aoust, Quebec (CA); Pierre-Olivier Lavoie, Quebec (CA); Louis-Philippe Vezina, Neuville (CA); Manon Couture, St. Augustin de Desmaures (CA)

(73) Assignee: MEDICAGO INC., Quebec, Quebec (CA)

(21) Appl. No.: 14/126,357

(22) PCT Filed: Jun. 13, 2012
(86) PCT No.: PCT/CA2012/000581

§ 371 Date: Mar. 20, 2014

Related U.S. Application Data
(60) Provisional application No. 61/496,371, filed on Jun. 13, 2011.
(60) Provisional application No. 61/578,787, filed on Dec. 21, 2011.

(51) Int. Cl. C07K 14/005

(52) U.S. Cl. CPC C07K 14/005
USPC 424/224.1; 435/69.1; 530/359

(57) ABSTRACT

A method of producing a virus like particle (VLP) in a plant is provided. The method comprises introducing a first nucleic acid into the plant, or portion of the plant. The first nucleic acid comprising a first regulatory region active in the plant operatively linked to a nucleotide sequence encoding a native rabies virus structural protein. The nucleotide sequence may further comprise one or more than one amplification element. Optionally, a second nucleic acid might be introduced into the plant, or portion of the plant. The second nucleic acid comprising a second regulatory region active in the plant and operatively linked to a nucleotide sequence encoding a matrix protein, for example but not limited to a rabies matrix protein. The plant or portion of the plant is incubated under conditions that permit the expression of the nucleic acids, thereby producing the VLP.

### FIELD OF INVENTION

The present invention relates to producing native viral proteins in plants. More specifically, the present invention also relates to producing virus-like particles comprising native rabies virus structural protein in plants.

### BACKGROUND OF THE INVENTION

Vaccination provides protection against disease caused by an infectious agent by inducing a subject to mount a defense prior to infection. Conventionally, this has been accomplished through the use of live attenuated or whole inactivated forms of the infectious agents as immunogens. To avoid the danger of using the whole virus (such as killed or attenuated viruses) as a vaccine, recombinant viral proteins, for example subunits, have been pursued as vaccines. Both peptide and subunit vaccines are subject to a number of potential limitations. Subunit vaccines may exhibit poor immunogenicity, owing to incorrect folding, poor antigen presentation, or differences in carbohydrate and lipid composition. A major problem is the difficulty of ensuring that the conformation of the engineered proteins mimics that of the antigens in their natural environment. Suitable adjuvants and, in the case of peptides, carrier proteins, must be used to boost the immune response. In addition these vaccines elicit primarily humoral responses, and thus may fail to evoke effective immunity. Subunit vaccines are often ineffective for diseases in which whole inactivated virus can be demonstrated to provide protection.

Virus-like particles (VLPs) are potential candidates for inclusion in immunogenic compositions. VLPs closely resemble mature virions, but they do not contain viral genomic material. Therefore, VLPs are nonreplicative in nature, which make them safe for administration as a vaccine. In addition, VLPs can be engineered to express viral glycoproteins on the surface of the VLP, which is their most native physiological configuration. Moreover, since VLPs resemble intact virions and are multivalent particulate structures, VLPs may be more effective in inducing neutralizing antibodies to the glycoprotein than soluble envelope protein antigens.

To date, VLPs have been produced for more than 30 different viruses that infect humans and other animals. One of the most striking features of this group is that it is extremely diverse in terms of the structure of the individual viruses. It includes viruses that have a single capsid protein, multiple capsid proteins, and those with and without lipid envelopes.

The formation of VLPs for viruses with a lipid envelope has a different type of technical challenge than those produced for viruses with multiple capsids. For these viruses, the choice of expression system can be important for the efficiency of VLP formation. For example, Hantaan virus readily forms VLPs when expressed in mammalian cells from a vaccinia-virus-based vector, but VLP formation is relatively inefficient in insect cells (Betenbaugh M et al. 1995, Virus Res. 38, 111-124).

The rabies virus (RV) is a member of the family Rhabdoviridae. Like most members of this family, RV is a non-segmented, negative stranded RNA virus whose genome codes for five viral proteins: RNA-dependent RNA polymerase (L); a nucleoprotein (N); a phosphorylated protein (P); a matrix protein (M) located on the inner side of the viral protein envelope; and an external surface glycoprotein (G). (Dietzschold B et al., 1991, Crit. Rev. Immunol. 10: 427-439.)

Cell-cultured based vaccines for rabies are limited to growing inactivated strains of the virus in cell cultures. These vaccines comprise the virus grown in cell cultures. Current biotechnological approaches aim at expressing the coat protein gene of the rabies virus to develop a safe recombinant protein that could be deployed as an active vaccine. Stable expression of rabies virus glycoprotein has been shown in Chinese Hamster Ovary cells (Burger et al., 1991, J Gen Virol., February; 72 (Pt 2):359-67). A full length, glycosylated protein of 67 K that co-migrated with the G-protein isolated from virus-infected cells, was obtained.

Expression of rabies glycoprotein gene by baculoviral vectors in insect cells gives yields of protein to the extent of 18% of total cellular protein, 48 h post infection. Prehaud D H et al, (1989, Virology, December; 173(2):390-9) describe placing a sequence encoding the G protein of CVS strain under control of the AcNPV polyhedrin promoter and expressing the construct using a Spodoptera fugiperda cell-line. The insect derived protein exhibited altered electrophoretic mobility compared to the wild type due to differences in the glycan components.

Rupprecht et al (1993, Vaccine, 11(9):925-8) demonstrate that a glycoprotein (ERA strain) derived from recombinant baculovirus-infected insect cells was efficacious as an oral vaccine in raccoons. WO/1993/001833 teaches the production of a virus like particles (VLPs) in a baculovirus expression system containing an RNA genome including a 3′ domain and a filler domain surrounded by a sheath of rabies N protein and rabies M protein. The VLP also includes a lipid envelope of rabies G protein. In view of relatively high costs of the insect and mammalian cell-systems these are not the systems of choice for G protein expression as a strategy to develop vaccine against rabies.

McGarvey et al. (1995, Bio/Technology Vol. 13, No. 13, pp. 1484-1487) describe the transformation of tomato cotyledons using a full length cDNA encoding glycoprotein G of rabies virus (ERA strain) under the control of the 35S′ promoter of cauliflower mosaic virus. The protein was expressed in tomato and was characterized as having a molecular weight of 62 and 60 kDa in western blot after immunoprecipitation, as compared to 66 kDa observed for G protein from virus grown in BHK cells. The difference in molecular weight compared to the natural glycoprotein was suggested to result from post-translational modification of the protein (proteolytic cleavage and/or modified glycosylation). The amount of G protein immunoprecipitated was found to be approximately 1-10 ng/mg of soluble protein i.e. at 0.0001% to 0.001% of soluble protein. The low expression level may have been due to using a poorly designed gene. For example, a native G protein coding gene was used along with its native signal peptide.

Plant derived immune response against diseases such as mink enteritis and rabies were reported by expressing viral epitopes on the surface of plant viruses, followed by infection of susceptible host with the recombinant modified virus (Modelska et al., 1998, Proc. Natl. Acad. Sci. USA 95: 2481-2485; Yusibov et al., 2002, Vaccine 20:3155-3164). The size of the antigen polypeptide expressed on surface of a vector virus was limited to 37 amino acids, and required epitope mapping of the antigen. Such thorough knowledge of the antigen is not generally available, especially with newly discovered diseases where the expression of full-length proteins may be the only option. I some cases, multiple epitopes may be required to give acceptable protection against challenge by the pathogenic virus. Furthermore, containment could be considered as a significant problem at the agricultural level, especially when environmentally stable plant viruses for example, Tobacco Mosaic Virus, are used.

WO 97/43428 teaches a method for producing the glycoprotein G of the rabies virus, or a virus related to the rabies virus, in plants. The construct comprised a sequence encoding a chimeric G protein comprising a mature viral protein G with a N-terminal signal peptide other than that naturally associated with the viral protein G. The glycoprotein had a molecular weight of approximately 66 kDa and was highly insoluble. Detergents, such as SDS, or Triton X-100, were necessary to extract and solubilize the glycoprotein. The authors concluded that the “insoluble” glycoprotein was related to the presence of the C-terminal transmembrane domain (the region located about 40 to 60 amino acids of the carboxy terminus of the glycoprotein) which is important for a protective response when the glycoprotein is used as vaccine.

Enveloped viruses may obtain their lipid envelope when ‘budding’ out of the infected cell and obtain the membrane from the plasma membrane, or from that of an internal organelle. For example, during the assembly process in rhabdoviruses, the N-P-L complex encapsulates negative-stranded genomic RNA to form the RNP core. The M protein forms a capsule, or matrix, around the RNP, and the RNP-M complex migrates to an area of the plasma membrane containing glycoprotein inserts. The M-protein initiates coiling, and the M-RNP complex binds with the glycoprotein, whereupon the completed virus buds from the plasma membrane.

Within the central nervous system (CNS), there is preferential viral budding from plasma membranes. Conversely, virus in the salivary glands buds primarily from the cell membrane into the acinar lumen. Viral budding into the salivary gland and virus-induced aggressive biting-behavior in the host animal maximize chances of viral infection of a new host. In mammalian or baculovirus cell systems, for example, rabies buds from the plasma membrane. Only a few enveloped viruses are known to infect plants (for example, members of the Topoviruses and Rhabdoviruses). Of the known plant enveloped viruses, they are characterized by budding from internal membranes of the host cell, and not from the plasma membrane. However, recombinant VLPs have been produced in plant hosts from the plasma membrane (WO 2011/035422; which is incorporated herein by reference).

Assembly/budding in rhabdoviruses is driven largely by the matrix (M) protein. The M protein contains a late budding domain that mediates the recruitment of host proteins linked to the vacuolar protein sorting pathway of the cell to facilitate virus-cell separation. Without wishing to be bound by theory, budding of enveloped viruses from cellular membranes is believed to depend on the presence of transmembrane spike proteins interacting with cytoplasmic virus components. For example, cells infected with rabies virus mutants that were deficient for the glycoprotein G, or the G cytoplasmic tail, released spikeless rhabdovirus particles, demonstrating that the viral surface protein is not required to drive the budding process. (Mebatsion T. et al., 1996, Cell, March 22:84(6):941-51). In contrast, infectious particles produced within M-protein deficient rabies virus mutants were mainly cell associated, and the yield of cell-free infectious virus was reduced by as much as 500,000-fold. This demonstrates the significant role of the M protein in virus budding. Supernatants from cells infected with the M-deficient rabies virus comprised long, rod-shaped virions, rather than the typical bullet-shaped rhabdovirus particles, further confirming impairment of the virus formation process. Complementation with M protein expressed from plasmids rescued rhabdovirus formation. The M protein therefore appears to play an important role in condensing and targeting the RNP to the plasma membrane as well as in incorporation of G protein into budding virions. (Mebatsion T. et al., 1999, J Virol January; 73(1):242-50).

### SUMMARY OF THE INVENTION

The present invention relates to producing native viral proteins in plants. More specifically, the present invention also relates to producing virus-like particles comprising native rabies virus structural protein in plants.

According to the present invention there is provided a method (A) of producing a rabies virus like particle (VLP) in a plant comprising,

a) introducing a first nucleic acid comprising a first regulatory region active in the plant operatively linked to a nucleotide sequence encoding a native rabies virus structural protein into the plant, or portion of the plant,

b) incubating the plant or portion of the plant under conditions that permit the expression of the nucleic acids, thereby producing the rabies VLP.

The native rabies virus structural protein may be a glycoprotein. If the native rabies virus structural protein is not an M protein, then the method (A) as described above may further comprise a step of:

c) introducing a second nucleic acid comprising a second regulatory region active in the plant and operatively linked to a nucleotide sequence encoding a matrix protein.

The first or second nucleotide sequence or both may further encode, comprise, or encode and comprise, one or more than one amplification element. The one or more than one amplification element may be selected from one or more than one geminivirus amplification element. The one or more than one geminivirus amplification element may be selected from a Bean Yellow Dwarf Virus long intergenic region (BeYDV LIR), and a BeYDV short intergenic region (BeYDV SIR).

The first regulatory region active in the plant, and the second regulatory region active in the plant may be the same or different.

The method as described above may further comprising a step of:

d) harvesting the plant and extracting the VLPs.

The present invention also includes the method (A) as described above, wherein the first nucleic acid sequence comprises the regulatory region operatively linked with a one or more than one comovirus enhancer, and a third nucleic acid encoding a suppressor of silencing, a geminivirus replicase, or both, are introduced into the plant or portion of the plant. Alternatively, first the nucleic acid sequence comprising the regulatory region operatively linked with a one or more than one comovirus enhancer, the second nucleic acid comprising a second regulatory region active in the plant and operatively linked to a nucleotide sequence encoding the matrix protein, and the third nucleic acid encoding a suppressor of silencing, a geminivirus replicase, or both, may be introduced into the plant or portion of the plant. If the third nucleic acid only comprises the suppressor of silencing, then a fourth nucleic acid encoding the geminivirus replicase may be introduced into the plant or portion of the plant. The one or more than one comovirus enhancer may be a comovirus UTR, for example, a Cowpea Mosaic Virus hyperanslatable (CPMV-HT) UTR such as the CPMV-HT 5′ and/or 3′UTR.

The present invention also includes the method (A) as described above, wherein in the step of introducing (step a), the first nucleic acid is transiently expressed in the plant. Alternatively, in the step of introducing (step a), the first nucleic acid is stably expressed in the plant.

The present invention also provides a method (B) of producing a rabies virus like particle (VLP) comprising,

a) providing a plant or portion of the plant comprising a first nucleic acid comprising a first regulatory region active in the plant operatively linked to a nucleotide sequence encoding one or more native rabies virus structural protein,

b) incubating the plant or portion of the plant under conditions that permit the expression of the nucleic acids, thereby producing the native rabies VLP.

The first regulatory region active in the plant, and the second regulatory region active in the plant may be the same or different.

The native rabies virus structural protein in method (B) may be a glycoprotein. If the a native rabies virus structural protein is not an M (matrix) protein, then in method (B) as described above the plant may further comprise a second nucleic acid comprising a second regulatory region active in the plant and operatively linked to a nucleotide sequence encoding a matrix protein. Alternatively, the first nucleic acid might comprise a nucleotide sequence encoding a matrix protein.

In the method as described above (Method B) the first nucleic acid or second nucleic acid or both may further encode, comprise, or encode and comprise, one or more than one amplification element. The one or more than one amplification element may be selected from one or more than one geminivirus amplification element. The one or more than one geminivirus amplification element may be selected from a Bean Yellow Dwarf Virus long intergenic region (BeYDV LIR), and a BeYDV short intergenic region (BeYDV SIR).

In the methods as described above (Methods A or B) the plant or portion of the plant may further comprise another nucleic acid sequence encoding a suppressor of silencing, for example HcPro or p19, a geminivirus replicase or both. Alternatively, the plant or portion of the plant may comprise yet another nucleic acid encoding the geminivirus replicase.

The present invention also includes the method (B) as described above, wherein the plant or portion of the plant transiently expressed the first nucleic acid. Alternatively, the first nucleic acid is stably expressed in the plant or portion of the plant.

The method (B) as described above may further comprising a step of:

d) harvesting the plant and extracting the VLPs.

The present invention provides a VLP produced by the methods (A) and/or (B) as described above. The VLP may further comprising one or more than one lipid derived from a plant. The, one or more native rabies virus structural protein comprises of the VLP may comprise plant-specific N-glycans, or modified N-glycans. The present invention also provides a polyclonal antibody prepared using the VLP.

The present invention includes a composition comprising an effective dose of the VLP made by the method (A) or (B) as just described, for inducing an immune response, and a pharmaceutically acceptable carrier.

The present invention also includes a method of inducing immunity to an rabies virus infection in a subject, comprising administering the VLP as just described, to the subject. The VLP may be administered to a subject orally, intradermally, intranasally, intramusclarly, intraperitoneally, intravenously, or subcutaneously.

The present invention also provides plant matter comprising a VLP produced by the method (A) and/or (B) described above. The plant matter may be used in inducing immunity to a rabies virus infection in a subject. The plant matter may also be admixed as a food supplement.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a Western blot analysis of transient rabies G protein expression in Nicotiana benthamiana. Rabies G protein was expressed under the control of CPMV-HT with (1091) or without (1071) BeYDV-based DNA amplification system (see table 2 in the Examples for constructs). Numbers in parenthesis refer to the amount of Agrobacterium culture, in milliliters, used in the preparation of the bacterial inoculum. Plants infiltrated with AGL1/1091 were harvest 3 or 4 days post-infiltration (DPI). Leaves of infiltrated plants were harvested and extracted mechanically. Protein extracts were separated by SDS-PAGE and analyzed by western blot using anti-rabies G mouse monoclonal antibodies (Santa-Cruz SC-57995).

FIG. 2 shows a comparison of rabies G protein content in protein extracts from biochemical and mechanical extraction methods for rabies G protein. Protein extracts were separated by SDS-PAGE and analyzed by western blot using anti-rabies G mouse monoclonal antibodies (Santa-Cruz SC-57995). Numbers in parenthesis refer to the amount of Agrobacterium culture, in milliliters, used in the preparation of the bacterial inoculum (see table 2 for constructs).

FIG. 3A shows a western blot analysis of rabies G protein content after separation, by size exclusion chromatography (SEC), of concentrated protein extracts from plants infiltrated with AGL1/1091. Elution fractions from SEC were separated by SDS-PAGE and analyzed by western blot using anti-rabies protein G mouse monoclonal antibodies (Santa-Cruz SC-57995). FIG. 3B shows a western blot analysis of rabies G protein content after separation, by size exclusion chromatography (SEC), of concentrated protein extracts from plants infiltrated with AGL1/1091+AGL1/1086. Elution fractions from SEC were separated by SDS-PAGE and analyzed by western blot using anti-rabies protein G mouse monoclonal antibodies (Santa-Cruz SC-57995).

FIG. 4A shows primer IF-RabM-S3.c (SEQ ID NO:1). FIG. 4B shows primer IF-RabM-S1−4.r (SEQ ID NO: 2). FIG. 4C shows synthesized M protein coding sequence (corresponding to nt 2496-3104 from Genbank accession number FJ913470) (SEQ ID NO. 3). FIG. 4D shows a schematic representation of construct number 1191. SacIl and StuI restriction enzyme sites used for plasmid linearization are annotated on the representation. FIG. 4E shows construct number 1191 from left to right t-DNA borders (underlined). 2×35S/CPMV-HT/NOS with Plastocyanin-P19-Plastocyanin expression cassette (suppressor of silencing) (SEQ ID NO: 4). FIG. 4F shows expression cassette number 1066 from 2×35S promoter to NOS terminator. The open reading frame of M protein from Rabies virus ERA strains is underlined (SEQ ID NO: 5). FIG. 4G shows the amino acid sequence of M protein from Rabies virus ERA strain (SEQ ID NO: 6). FIG. 4H shows the schematic representation of construct number 1066.

FIG. 5A shows a schematic representation of construct number 1193. SacII and StuI restriction enzyme sites used for plasmid linearization are annotated on the representation. FIG. 5B shows construct number 1193 from left to right t-DNA borders (underlined). 2×35S/CPMV-HT/NOS into BeYDV+Replicase amplification system with Plastocyanin-TBSV P19-Plastocyanin expression cassette (suppressor of silencing) (SEQ ID NO:7). FIG. 5C shows expression cassette number 1086 from BeYDV left LIR to BeYDV right LIR. Open reading frame of PDISP/G protein from Rabies virus ERA strain is underlined. (SEQ ID NO: 8). FIG. 5D shows a schematic representation of construct number 1086.

FIG. 6A shows primer IF-RabG-S2+4.c (SEQ ID NO: 9). FIG. 6B shows primer IF-RabG-S1−4.r (SEQ ID NO:10). FIG. 6C shows synthesized Rabies G protein coding sequence (corresponding to nt 3317-4891 from Genbank accession number EF206707) (SEQ ID NO: 11). FIG. 6D shows a schematic representation of construct number 1192. SacII and StuI restriction enzyme sites used for plasmid linearization are annotated on the representation. FIG. 6E shows construct number 1192 from left to right t-DNA borders (underlined). 2×35S/CPMV-HT/PDISP/NOS with Plastocyanin-P19-Plastocyanin expression cassette (suppressor of silencing) (SEQ ID NO: 12). FIG. 6F shows expression cassette number 1071 from 2×35S promoter to NOS terminator. Open reading frame of PDISP/G protein from Rabies virus ERA strain is underlined. (SEQ ID NO: 13). FIG. 6G shows amino acid sequence of PDISP-G protein from Rabies virus ERA strain (SEQ ID NO: 14). FIG. 6H shows a schematic representation of construct number 1071.

FIG. 7A shows a schematic representation of construct number 1194. SacII and StuI restriction enzyme sites used for plasmid linearization are annotated on the representation. FIG. 7B shows construct number 1194 from left to right t-DNA borders (underlined). 2×35S/CPMV-HT/PDISP/NOS into BeYDV+Replicase amplification system with Plastocyanin-P19-Plastocyanin expression cassette (suppressor of silencing) (SEQ ID NO: 15). FIG. 7C shows expression cassette number 1091 from BeYDV left LIR to BeYDV right LIR. Open reading frame of PDISP/G protein from Rabies virus ERA strain is underlined. (SEQ ID NO:16). FIG. 7D shows a schematic representation of construct number 1091.

FIG. 8A shows a coomassie stained SDS-PAGE (Precast 4-12%, from BioRad) showing G protein VLP preparation. 1) Rab-VLP preparation, 2) molecular weight marker. FIG. 8B shows responses of neutralizing antibodies (IU/ml) in groups of mice (5 per group) immunized intramuscularly (i.m.) with three doses (D0, D7 and D28) of 0.1 ml of the NG-VLP (Native G protein VLP) vaccine with or without adjuvant (Alhydrogel (Alhy)). Blood samples were taken on Day 44, 16 days after the 3rd doses. Method of testing used: RFFIT test (rapid fluorescent focus inhibition test): Geometric Mean Titers (GMT) was calculated, using the individual values obtained for each animal and positive responders are indicated. Bars represent GMT with 95% CI. Anova was performed between all treatment groups and no statistically significant differences were reported.

### DETAILED DESCRIPTION

The following description is of a preferred embodiment.

The present invention relates to virus-like particles (VLPs) comprising one or more native rabies virus structural protein, and methods of producing rabies VLPs in plants. The rabies VLPs may comprise one or more native rabies virus structural protein, for example a one or more glycoprotein, one or more matrix protein, or both. The VLP does not comprise virus proteins from a plant virus.

The present invention in part provides a method of producing a rabies virus like particle (VLP) in a plant. The method may comprise introducing a nucleic acid comprising a regulatory region active in the plant operatively linked to a nucleotide sequence encoding a native rabies virus structural protein and one or more than one amplification element, into the plant, or portion of the plant. Followed by incubating the plant or portion of the plant under conditions that permit the expression of the nucleic acids, thereby producing the VLP.

The native rabies structural virus protein (also referred to as native rabies structural viral protein) may refer to all or a portion of an native rabies virus structural protein sequence isolated from rabies virus, present in any naturally occurring or variant rabies virus strain or isolate. Thus, the term native rabies virus structural protein and the like include naturally occurring native rabies virus structural protein sequence variants produced by mutation during the virus life-cycle or produced in response to selective pressure (e.g., drug therapy, expansion of host cell tropism or infectivity, etc.). As one of skill in the art appreciates, such native rabies virus structural protein sequences and variants thereof may be also produced using recombinant techniques. The native rabies structural virus protein does not include chimeric proteins wherein for example a transmembrane domain and/or a cytoplasmic tail have been replaced with a heterologous transmembrane domain and/or a cytoplasmic tail with respect to the native rabies structural virus protein.

A non-limiting example of a native rabies virus structural protein is rabies glycoprotein (G) protein, a fragment of G protein, a matrix (M) protein, a fragment of M protein, or a combination thereof. Non-limiting examples of G protein, or fragments of G protein that may be used according to the present invention include those G protein from rabies ERA strain. An example of a G protein, which is not to be considered limiting, is set forth in the amino acid sequence of SEQ ID NO: 14. Furthermore, the native rabies structural virus protein may comprise the sequence set forth in SEQ ID NO: 14, or a sequence having at least about 90-100% sequence similarity thereto, including any percent similarity within these ranges, such as 91, 92, 93, 94, 95, 96, 97, 98, 99% sequence similarity thereto.

Amino acid sequence similarity or identity may be computed by using the BLASTP and TBLASTN programs which employ the BLAST (basic local alignment search tool) 2.0 algorithm. Techniques for computing amino acid sequence similarity or identity are well known to those skilled in the art, and the use of the BLAST algorithm is described in ALTSCHUL et al. (1990, J Mol. Biol. 215: 403-410) and ALTSCHUL et al. (1997, Nucleic Acids Res. 25: 3389-3402).

The native structural viral protein may exist as a monomer, a dimer, a trimer, or a combination thereof. A trimer is a macromolecular complex formed by three, usually non-covalently bound proteins. Without wishing to be bound by theory, the trimerization domain of a protein may be important for the formation such trimers. Therefore the viral protein or fragment thereof may comprise a trimerization domain.

By “matrix protein” (also referred to as viral core protein) it is meant a protein that that organizes and maintains virion structure. Viral matrix proteins usually interact directly with cellular membranes and can be involved in the budding process. Viral core proteins are proteins that make up part of the nucelocapsid and typically are directly associated with the viral nucleic acid. Examples of viral matrix or core protein are rabies M protein, influenza M1, RSV M and retrovirus gag proteins. Examples of Matrix proteins that may be used as described herein include, but are not limited to rabies M protein. Non-limiting example of sequences that may be used with the present invention include M protein from rabies virus ERA strain. An exemplary M protein consists of the amino acid sequence as shown in SEQ ID NO: 6. Further more, the native rabies structural virus protein may comprise the sequence set forth in SEQ ID NO: 6 or sequences having at least about 90-100% sequence similarity thereto, including any percent similarity within these ranges, such as 91, 92, 93, 94, 95, 96, 97, 98, 99% sequence similarity thereto.

Both vesicular stomatitis virus (a rhabdovirus like rabies) and Herpes Simplex virus (a herpes virus like varicella-zoster virus) bud in a VSP4-dependent manner (Taylor et al. J. Virol 81:13631-13639, 2007; Crump et al., J. Virol 81:7380-7387, 2007). Since VSP4 interacts with the late domain of the matrix protein, this suggests that the matrix protein is required for budding and, as a corollary, for VLP production. However, as described herein, native rabies VLPs may be produced within plants with or without co-expression of a matrix protein. Therefore, native rabies VLPs produced in plants from virus derived native structural proteins, in accordance with the present invention may or may not comprise viral matrix (or viral core) protein.

The present invention also provides for a method of producing rabies virus like protein VLPs in a plant, wherein a nucleic acid (a first nucleic acid) encoding a native rabies virus structural protein, for example a rabies G protein, is co-expressed with a second nucleic acid encoding a viral matrix protein, for example but not limited to rabies matrix protein. The nucleic acid, and second nucleic acid, may be introduced to the plant in the same step, or may be introduced to the plant sequentially.

As described in more detail below, VLPs may be produced in a plant by expressing a nucleic acid (a first nucleic acid) encoding one or more native rabies virus structural protein, for example a rabies G protein. A second nucleic acid encoding a matrix protein, for example but not limited to rabies matrix protein might be co-expressed in the plant. The nucleic acid and second nucleic acid may be introduced to the plant in the same step, or they may be introduced to the plant sequentially. The nucleic acid and second nucleic acid may be introduced in the plant in a transient manner, or in a stably manner.

Furthermore, a plant that expresses a first nucleic acid encoding one or more native rabies virus structural protein, for example a rabies G protein, may be transformed with a matrix protein, for example but not limited to a rabies matrix protein, (second nucleic acid) so that both the first and the second nucleic acids are co-expressed in the plant. Alternatively, a plant that expresses a matrix protein, for example but not limited to a rabies matrix protein, (second nucleic acid) may be transformed with a first nucleic acid encoding one or more native rabies virus structural protein, for example a rabies G protein, so that both the first and the second nucleic acids are co-expressed in the plant.

Additionally, a first plant expressing the first nucleic acid encoding one or more native rabies virus structural protein, for example a rabies G protein, may be crossed with a second plant expressing the second nucleic acid encoding the matrix protein for example but not limited to rabies matrix protein, to produce a progeny plant that co-expresses the first and second nucleic acids encoding the native rabies virus structural protein and the matrix protein, respectively.

The present invention also provides a method of producing rabies virus VLPs in a plant that involves introducing one or more nucleic acid encoding one or more native rabies virus structural protein operatively linked to a regulatory region active in the plant, and one or more than one amplification elements, into the plant or portion of the plant. The plant or portion of the plant is then incubated under conditions that permit the expression of the one or more nucleic acid, thereby producing the rabies virus VLPs. The one or more native rabies virus structural protein may be one or more rabies G protein, a fragment of the G protein, one or more M protein, a fragment of the M protein, or a combination thereof.

The present invention further provides for a VLP comprising one or more native rabies virus structural protein for example but not limited to one or more rabies virus glycoprotein, one or more matrix protein or both. The VLP may be produced by the method as provided by the present invention.

The term “virus like particle” (VLP), or “virus-like particles” or “VLPs” refers to structures that self-assemble and comprise one or more structural proteins such as for example native rabies virus structural protein for example but not limited to rabies G protein. VLPs are generally morphologically and antigenically similar to virions produced in an infection, but lack genetic information sufficient to replicate and thus are non-infectious. VLPs may be produced in suitable host cells including plant host cells. Following extraction from the host cell and upon isolation and further purification under suitable conditions, VLPs may be purified as intact structures.

The size (i.e. the diameter) of the above-defined VLPs, maybe measures for example by dynamic light scattering (DLS) or electron microscope (EM) techniques, is usually between 40 to 300 nm, or any size therebetween. In some embodiments, the size of the intact VLP structure may range from about 40 nm to about 300 nm, or any size therebetween, such as 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm or 280 nm, or any size therebetween. In an embodiment the size of the intact VLP structure may range from about 170 nm to about 200 nm or any size therebetween, such as 175 nm, 180 nm, 185 nm, 190 nm, 195 nm or any size therebetween.

The present invention further provides a nucleic acid comprising a nucleotide sequence encoding one or more native rabies virus structural protein operatively linked to a regulatory region active in a plant. Furthermore one or more native rabies virus structural protein may be operatively linked to one or more than one amplification elements. The one or more native rabies virus structural protein may be for example one or more rabies G protein one or more M protein, or both.

A nucleic acid sequence referred to in the present invention, may be “substantially homologous” or “substantially similar” to a sequence, or a compliment of the sequence if the nucleic acid sequence hybridise to one or more than one nucleotide sequence or a compliment of the nucleic acid sequence as defined herein under stringent hybridisation conditions. Sequences are “substantially homologous” “substantially similar” when at least about 70%, or between 70 to 100%, or any amount therebetween, for example 70, 72, 74, 76, 78, 80, 82, 84, 86, 88, 90, 92, 94, 96, 98, 100%, or any amount therebetween, of the nucleotides match over a defined length of the nucleotide sequence providing that such homologous sequences exhibit one or more than one of the properties of the sequence, or the encoded product as described herein. Correct folding of the protein may be important for stability of the protein, formation of multimers, formation of VLPs and function. Folding of a protein may be influenced by one or more factors, including, but not limited to, the sequence of the protein, the relative abundance of the protein, the degree of intracellular crowding, the availability of cofactors that may bind or be transiently associated with the folded, partially folded or unfolded protein.

Such a sequence similarity may be determined using a nucleotide sequence comparison program, such as that provided within DNASIS (using, for example but not limited to, the following parameters: GAP penalty 5, # of top diagonals 5, fixed GAP penalty 10, k-tuple 2, floating gap 10, and window size 5). However, other methods of alignment of sequences for comparison are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (GAP, BESTFIT, FASTA, and BLAST, available through the NIH.), or by manual alignment and visual inspection (see, e.g., Current Protocols in Molecular Biology, Ausubel et al., eds. 1995 supplement), or using Southern or Northern hybridization under stringent conditions (see Maniatis et al., in Molecular Cloning (A Laboratory Manual), Cold Spring Harbor Laboratory, 1982). Preferably, sequences that are substantially homologous exhibit at least about 80% and most preferably at least about 90% sequence similarity over a defined length of the molecule.

An example of one such stringent hybridization conditions may be overnight (from about 16-20 hours) hybridization in 4×SSC at 65° C., followed by washing in 0.1×SSC at 65° C. for an hour, or 2 washes in 0.1×SSC at 65° C. each for 20 or 30 minutes. Alternatively an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours), or hybridization in Church aqueous phosphate buffer (7% SDS; 0.5M NaPO₄ buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each for unique sequence regions.

A nucleic acid encoding a native rabies structural polypeptide or a native rabies virus structural protein may be described as a “rabies nucleic acid”, a “rabies nucleotide sequence”, a “native rabies nucleic acid”, or a “native rabies nucleotide sequence”. For example, which is not to be considered limiting, a virus-like particle comprising one or more native rabies virus structural protein or native rabies virus structural polypeptide, may be described as a “rabies VLP” or “native rabies VLP”.

The native rabies virus structural protein or polypeptide may include a signal peptide that is the same as, or heterologous with, the remainder of the polypeptide or protein. The term “signal peptide” is well known in the art and refers generally to a short (about 5-30 amino acids) sequence of amino acids, found generally at the N-terminus of a polypeptide that may direct translocation of the newly-translated polypeptide to a particular organelle, or aid in positioning of specific domains of the polypeptide chain relative to others. As a non-limiting example, the signal peptide may target the translocation of the protein into the endoplasmic reticulum and/or aid in positioning of the N-terminus proximal domain relative to a membrane-anchor domain of the nascent polypeptide to aid in cleavage and folding of the mature protein, for example which is not to be considered limiting, a native rabies virus structural protein.

A signal peptide (SP) may be native to the protein or virus protein, or a signal peptide may be heterologous with respect to the primary sequence of the protein or virus protein being expressed. For example the native signal peptide of native rabies G protein or native rabies M protein may be used to express the native rabies virus structural protein in a plant system.

A signal peptide may also be non-native, for example, from a protein, viral protein or native structural protein of a virus other than virus protein, or from a plant, animal or bacterial polypeptide. A non limiting example of a signal peptide that may be used is that of alfalfa protein disulfide isomerase (PDI SP; nucleotides 32-103 of Accession No. Z11499).

The present invention therefore provides for a native rabies virus structural protein comprising a native, or a non-native signal peptide, and nucleic acids encoding such rabies virus structural proteins.

### Amplification Element

The native rabies virus structural protein or polypeptide may be expressed in an expression system comprising a viral based, DNA or RNA, expression system, for example but not limited to, a comovirus-based expression cassette and geminivirus-based amplification element.

The expression system as described herein may comprise an expression cassette based on a bipartite virus, or a virus with a bipartite genome. For example, the bipartite viruses may be of the Comoviridae family. Genera of the Comoviridae family include Comovirus, Nepovirus, Fabavirus, Cheravirus and Sadwavirus. Comoviruses include Cowpea mosaic virus (CPMV), Cowpea severe mosaic virus (CPSMV), Squash mosaic virus (SqMV), Red clover mottle virus (RCMV), Bean pod mottle virus (BPMV), Turnip ringspot virus (TuRSV), Broad bean true mosaic virus (BBtMV), Broad bean stain virus (BBSV), Radish mosaic virus (RaMV). Examples of comoviruse RNA-2 sequences comprising enhancer elements that may be useful for various aspects of the invention include, but are not limited to: CPMV RNA-2 (GenBank Accession No. NC_{—}003550), RCMV RNA-2 (GenBank Accession No. NC_{—}003738), BPMV RNA-2 (GenBank Accession No. NC_{—}003495), CPSMV RNA-2 (GenBank Accession No. NC_{—}003544), SqMV RNA-2 (GenBank Accession No. NC_{—}003800), TuRSV RNA-2 (GenBank Accession No. NC_{—}013219.1). BBtMV RNA-2 (GenBank Accession No. GU810904), BBSV RNA2 (GenBank Accession No. FJ028650), RaMV (GenBank Accession No. NC_{—}003800).

Segments of the bipartite comoviral RNA genome are referred to as RNA-1 and RNA-2. RNA-1 encodes the proteins involved in replication while RNA-2 encodes the proteins necessary for cell-to-cell movement and the two capsid proteins. Any suitable comovirus-based cassette may be used including CPMV, CPSMV, SqMV, RCMV, or BPMV, for example, the expression cassette may be based on CPMV.

“Expression cassette” refers to a nucleotide sequence comprising a nucleic acid of interest under the control of, and operably (or operatively) linked to, an appropriate promoter or other regulatory elements for transcription of the nucleic acid of interest in a host cell.

The expression systems may also comprise amplification elements from a geminivirus for example, an amplification element from the bean yellow dwarf virus (BeYDV). BeYDV belongs to the Mastreviruses genus adapted to dicotyledonous plants. BeYDV is monopartite having a single-strand circular DNA genome and can replicate to very high copy numbers by a rolling circle mechanism. BeYDV-derived DNA replicon vector systems have been used for rapid high-yield protein production in plants.

As used herein, the phrase “amplification elements” refers to a nucleic acid segment comprising at least a portion of one ore more long intergenic regions or long intergenic repeat (LIR) of a geminivirus genome. As used herein, “long intergenic region” or “long intergenic repeat” refers to a region of a long intergenic region that contains a rep binding site capable of mediating excision and replication by a geminivirus Rep protein. In some aspects, the nucleic acid segment comprising one or more LIRs, may further comprises a short intergenic region or small intergenic region (SIR) of a geminivirus genome. As used herein, “short intergenic region” or “small intergenic region” refers to the complementary strand (the short IR (SIR) of a Mastreviruses). Any suitable geminivirus-derived amplification element may be used herein. See, for example, WO2000/20557; WO2010/025285; Zhang X. et al. (2005, Biotechnology and Bioengineering, Vol. 93, 271-279), Huang Z. et al. (2009, Biotechnology and Bioengineering, Vol. 103, 706-714), Huang Z. et al. (2009, Biotechnology and Bioengineering, Vol. 106, 9-17); which are herein incorporated by reference). If more than one LIR is used in the construct, for example two LIRs, then the promoter, CMPV-HT regions and the nucleic acid sequence of interest and the terminator are bracketed by each of the two LIRs. Furthermore, the amplification element might for example originate from the sequence as disclosed in Halley-Stott et al. (2007) Archives of Virology 152: 1237-1240, deposited under Gen Bank accession number DQ458791, which are herein incorporated by reference. The nucleic acid segment comprising LIRs are joined nucleotides 2401 to 2566 and 1 to 128. The nucleic acid segment comprising SIRs are nucleotides 1154 to 1212.

As described herein, co-delivery of bean yellow dwarf virus (BeYDV)-derived vector and a Rep/RepA-supplying vector, by agroinfiltration of Nicotiana benthamiana leaves results in efficient replicon amplification and robust protein production.

A comovirus-based expression cassette and a geminivirus-derived amplification element may be comprised on separate vectors, or the component parts may be included in one vector. If two vectors are used, the first and second vectors may be introduced into a plant cell simultaneously, or separately.

A viral replicase may also be included in the expression system as described herein to increase expression of the nucleic acid of interest. An non-limiting example of a replicase is a BeYDV replicase (pREP110) encoding BeYDV Rep and RepA (C2/C1; Huang et al., 2009, Biotechnol. Bioeng. 103, 706-714; which is incorporated herein by reference). Another non-limiting example of a replicase is disclosed in Halley-Stott et al. (2007) Archives of Virology 152: 1237-1240, deposited under Gen Bank accession number DQ458791, which are herein incorporated by reference. The nucleic acid segment comprising C1:C2 gene are nucleotides 1310 to 2400.

By “co-expressed” it is meant that two or more than two nucleotide sequences are expressed at about the same time within the plant, and within the same tissue of the plant. However, the nucleotide sequences need not be expressed at exactly the same time. Rather, the two or more nucleotide sequences are expressed in a manner such that the encoded products have a chance to interact. The two or more than two nucleotide sequences can be co-expressed using a transient expression system, where the two or more sequences are introduced within the plant at about the same time under conditions that both sequences are expressed. Alternatively, a platform plant comprising one of the nucleotide sequences may be transformed in a stable manner, with an additional sequence encoding the protein of interest for example the native rabies virus structural protein, introduced into the platform plant in a transient manner.

### Chaperon

Correct folding of the expressed native rabies virus structural protein may be important for stability of the protein, formation of multimers, formation of VLPs, function of the native rabies virus structural protein and recognition of the native rabies virus structural protein by an antibody, among other characteristics. Folding and accumulation of a protein may be influenced by one or more factors, including, but not limited to, the sequence of the protein, the relative abundance of the protein, the degree of intracellular crowding, the pH in a cell compartment, the availability of cofactors that may bind or be transiently associated with the folded, partially folded or unfolded protein, the presence of one or more chaperone proteins, or the like.

Heat shock proteins (Hsp) or stress proteins are examples of chaperone proteins, which may participate in various cellular processes including protein synthesis, intracellular trafficking, prevention of misfolding, prevention of protein aggregation, assembly and disassembly of protein complexes, protein folding, and protein disaggregation. Examples of such chaperone proteins include, but are not limited to, Hsp60, Hsp65, Hsp 70, Hsp90, Hsp100, Hsp20-30, Hsp10, Hsp100-200, Hsp100, Hsp90, Lon, TF55, FKBPs, cyclophilins, ClpP, GrpE, ubiquitin, calnexin, and protein disulfide isomerases (see, for example, Macario, A. J. L., Cold Spring Harbor Laboratory Res. 25:59-70. 1995; Parsell, D. A. & Lindquist, S. Ann. Rev. Genet. 27:437-496 (1993); U.S. Pat. No. 5,232,833). As described herein, chaperone proteins, for example but not limited to Hsp40 and Hsp70 may be used to ensure folding of a rabies virus protein.

Examples of Hsp70 include Hsp72 and Hsc73 from mammalian cells, DnaK from bacteria, particularly mycobacteria such as Mycobacterium leprae, Mycobacterium tuberculosis, and Mycobacterium bovis (such as Bacille-Calmette Guerin: referred to herein as Hsp71). DnaK from Escherichia coli, yeast and other prokaryotes, and BiP and Grp78 from eukaryotes, such as A. thaliana (Lin et al. 2001 (Cell Stress and Chaperones 6:201-208). A particular example of an Hsp70 is A. thaliana Hsp70 (encoded by Genbank ref: AY120747.1). Hsp70 is capable of specifically binding ATP as well as unfolded polypeptides and peptides, thereby participating in protein folding and unfolding as well as in the assembly and disassembly of protein complexes.

Examples of Hsp40 include DnaJ from prokaryotes such as E. coli and mycobacteria and HSJ1, HDJ1 and Hsp40 from eukaryotes, such as alfalfa (Frugis et al., 1999. Plant Molecular Biology 40:397-408). A particular example of an Hsp40 is M. sativa MsJ1 (Genbank ref: AJ000995.1). Hsp40 plays a role as a molecular chaperone in protein folding, thermotolerance and DNA replication, among other cellular activities.

Among Hsps, Hsp70 and its co-chaperone, Hsp40, are involved in the stabilization of translating and newly synthesized polypeptides before the synthesis is complete. Without wishing to be bound by theory, Hsp40 binds to the hydrophobic patches of unfolded (nascent or newly transferred) polypeptides, thus facilitating the interaction of Hsp70-ATP complex with the polypeptide. ATP hydrolysis leads to the formation of a stable complex between the polypeptide, Hsp70 and ADP, and release of Hsp40. The association of Hsp70-ADP complex with the hydrophobic patches of the polypeptide prevents their interaction with other hydrophobic patches, preventing the incorrect folding and the formation of aggregates with other proteins (reviewed in Hartl, F U. 1996. Nature 381:571-579).

Native chaperone proteins may be able to facilitate correct folding of low levels of recombinant protein, but as the expression levels increase, the abundance of native chaperones may become a limiting factor. High levels of expression of native rabies virus structural protein in the agroinfiltrated leaves may lead to the accumulation of native rabies virus structural protein in the cytosol, and co-expression of one or more than one chaperone proteins such as Hsp70, Hsp40 or both Hsp70 and Hsp40 may reduce the level of misfolded or aggregated proteins, and increase the number of proteins exhibiting tertiary and quaternary structural characteristics that allow for formation of virus-like particles.

Therefore, the present invention also provides for a method of producing rabies VLPs in a plant, wherein a first nucleic acid encoding a native rabies virus structural protein is co-expressed with a second nucleic acid encoding a chaperone. The first and second nucleic acids may be introduced to the plant in the same step, or may be introduced to the plant sequentially.

### Regulatory Element

The use of the terms “regulatory region”, “regulatory element” or “promoter” in the present application is meant to reflect a portion of nucleic acid typically, but not always, upstream of the protein coding region of a gene, which may be comprised of either DNA or RNA, or both DNA and RNA. When a regulatory region is active, and in operative association, or operatively linked, with a gene of interest, this may result in expression of the gene of interest. A regulatory element may be capable of mediating organ specificity, or controlling developmental or temporal gene activation. A “regulatory region” may includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, may also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element comprises a basal promoter element, responsible for the initiation of transcription, as well as other regulatory elements (as listed above) that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152: 595-599; Bilodeau et al., 1994, Plant Cell 14: 125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, L R. P., 1998, Trends Plant Sci. 3, 352-358; which is incorporated by reference). Examples, of potential inducible promoters include, but not limited to, tetracycline-inducible promoter (Gatz, C., 1997, Ann. Rev. Plant Physiol. Plant Mol. Biol. 48, 89-108; which is incorporated by reference), steroid inducible promoter (Aoyama. T. and Chua, N. H., 1997, Plant 1. 2, 397-404; which is incorporated by reference) and ethanol-inducible promoter (Salter, M. G., et al, 1998, Plant 10urnal 16, 127-132; Caddick, M. X., et al, 1998, Nature Biotech. 16, 177-180, which are incorporated by reference) cytokinin inducible IB6 and CKI 1 genes (Brandstatter, I. and K.ieber, 1.1., 1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

A constitutive regulatory region directs the expression of a gene throughout the various parts of a plant and continuously throughout plant development. Examples of known constitutive regulatory elements include promoters associated with the CaMV ³⁵S transcript (Odell et al., 1985, Nature, 313: 810-812), the rice actin 1 (Zhang et al, 1991, Plant Cell, 3: 1155-1165), actin 2 (An et al., 1996, Plant J., 10: 107-121), or tms 2 (U.S. Pat. No. 5,428,147, which is incorporated herein by reference), and triosephosphate isomerase 1 (Xu et. al., 1994, Plant Physiol. 106: 459-467) genes, the maize ubiquitin 1 gene (Cornejo et ai, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995, Plant Mol. Biol. 29: 995-1004).

The term “constitutive” as used herein does not necessarily indicate that a gene under control of the constitutive regulatory region is expressed at the same level in all cell types, but that the gene is expressed in a wide range of cell types even though variation in abundance is often observed. Constitutive regulatory elements may be coupled with other sequences to further enhance the transcription and/or translation of the nucleotide sequence to which they are operatively linked. For example, the CPMV-HT system is derived from the untranslated regions of the Cowpea mosaic virus (CPMV) and demonstrates enhanced translation of the associated coding sequence. By “native” it is meant that the nucleic acid or amino acid sequence is naturally occurring, or “wild type”. By “operatively linked” it is meant that the particular sequences, for example a regulatory element and a coding region of interest, interact either directly or indirectly to carry out an intended function, such as mediation or modulation of gene expression. The interaction of operatively linked sequences may, for example, be mediated by proteins that interact with the operatively linked sequences.

The invention also provides VLPs that obtain a lipid envelope from the plasma membrane of the cell in which the VLPs are expressed. For example, if the one or more native rabies virus structural protein is expressed in a plant-based system, the resulting VLP may obtain a lipid envelope from the plasma membrane of the plant cell.

Generally, the term “lipid” refers to a fat-soluble (lipophilic), naturally-occurring molecule. A VLP produced in a plant according to some aspects of the invention may be complexed with plant-derived lipids. The plant-derived lipids may be in the form of a lipid bilayer, and may further comprise an envelope surrounding the VLP. The plant-derived lipids may comprise lipid components of the plasma membrane of the plant where the VLP is produced, including phospholipids, tri-, di- and monoglycerides, as well as fat-soluble sterol or metabolites comprising sterols. Examples include phosphatidylcholine (PC), phosphatidylethanolamine (PE), phosphatidylinositol, phosphatidylserine, glycosphingolipids, phytosterols or a combination thereof. A plant-derived lipid may alternately be referred to as a ‘plant lipid’. Examples of phytosterols include campesterol, stigmasterol, ergosterol, brassicasterol, delta-7-stigmasterol, delta-7-avenasterol, daunosterol, sitosterol, 24-methylcholesterol, cholesterol or beta-sitosterol—see, for example, Mongrand et ai., 2004. As one of skill in the art would understand, the lipid composition of the plasma membrane of a cell may vary with the culture or growth conditions of the cell or organism, or species, from which the cell is obtained. Generally, beta-sitosterol is the most abundant phytosterol.

Cell membranes generally comprise lipid bilayers, as well as proteins for various functions. Localized concentrations of particular lipids may be found in the lipid bilayer, referred to as ‘lipid rafts’. These lipid raft microdomains may be enriched in sphingolipids and sterols. Without wishing to be bound by theory, lipid rafts may have significant roles in endo and exocytosis, entry or egress of viruses or other infectious agents, inter-cell signal transduction, interaction with other structural components of the cell or organism, such as intracellular and extracellular matrices.

The VLP produced within a plant may induce a native rabies virus structural protein comprising plant-specific N-glycans. Therefore, this invention also provides for a VLP comprising native rabies virus structural protein having plant specific N-glycans.

Furthermore, modification of N-glycan in plants is known (see for example U.S. 60/944,344; which is incorporated herein by reference) and native rabies virus structural proteins having modified N-glycans may be produced. Native rabies virus structural proteins comprising a modified glycosylation pattern, for example with reduced fucosylated, xylosylated, or both, fucosylated and xylosylated, N-glycans may be obtained, or native rabies virus structural proteins having a modified glycosylation pattern may be obtained, wherein the protein lacks fucosylation, xylosylation, or both, and comprises increased galatosylation. Furthermore, modulation of post-translational modifications, for example, the addition of terminal galactose may result in a reduction of fucosylation and xylosylation of the expressed native rabies virus structural proteins when compared to a wild-type plant expressing native rabies virus structural proteins.

For example, which is not to be considered limiting, the synthesis of native rabies virus structural proteins having a modified glycosylation pattern may be achieved by co-expressing the native rabies virus structural protein along with a nucleotide sequence encoding beta-1.4 galactosyltransferase (GalT), for example, but not limited to mammalian GalT, or human GalT however GalT from another sources may also be used. The catalytic domain of GalT may also be fused to a CTS domain (i.e. the cytoplasmic tail, transmembrane domain, stem region) of N-acetylglucosaminyl transferase (GNT1), to produce a GNT1-GalT hybrid enzyme, and the hybrid enzyme may be co-expressed with native rabies virus structural protein. The native rabies virus structural protein may also be co-expressed along with a nucleotide sequence encoding N-acetylglucosaminyltransferase III (GnT-III), for example but not limited to mammalian GnT-III or human GnT-III, GnT-III from other sources may also be used. Additionally, a GNT1-GnT-III hybrid enzyme, comprising the CTS of GNT1 fused to GnT-III may also be used.

Therefore the present invention also provides VLP's comprising native rabies virus structural protein having modified N-glycans.

Without wishing to be bound by theory, the presence of plant N-glycans on native rabies virus structural protein may stimulate the immune response by promoting the binding of native rabies virus structural protein by antigen presenting cells. Stimulation of the immune response using plant N glycan has been proposed by Saint-Jore-Dupas et al. (2007). Furthermore, the conformation of the VLP may be advantageous for the presentation of the antigen, and enhance the adjuvant effect of VLP when complexed with a plant derived lipid layer.

The occurrence of VLPs may be detected using any suitable method for example, sucrose gradients, or size exclusion chromatography. VLPs may be assessed for structure and size by, for example electron microscopy, or by size exclusion chromatography.

For size exclusion chromatography, total soluble proteins may be extracted from plant tissue by homogenizing (Polytron) sample of frozen-crushed plant material in extraction buffer, and insoluble material removed by centrifugation. Precipitation with ice cold acetone or PEG may also be of benefit. The soluble protein is quantified, and the extract passed through a Sephacryl™ column, for example a Sephacryl™ S500 column. Blue Dextran 2000 may be used as a calibration standard. Following chromatography, fractions may be further analyzed by immunoblot to determine the protein complement of the fraction.

The separated fraction may be for example a supernatant (if centrifuged, sedimented, or precipitated), or a filtrate (if filtered), and is enriched for proteins, or suprastructure proteins, such as for example rosette-like structures or higher-order, higher molecular weight, particles such as VLPs. The separated fraction may be further processed to isolate, purify, concentrate or a combination thereof, the proteins, or suprastructure proteins, by, for example, additional centrifugation steps, precipitation, chromatographic steps (e.g. size exclusion, ion exchange, affinity chromatography), tangential flow filtration, or a combination thereof. The presence of purified proteins, or suprastructure proteins, may be confirmed by, for example, native or SDS-PAGE, Western analysis using an appropriate detection antibody, capillary electrophoresis, electron microscopy, or any other method as would be evident to one of skill in the art.

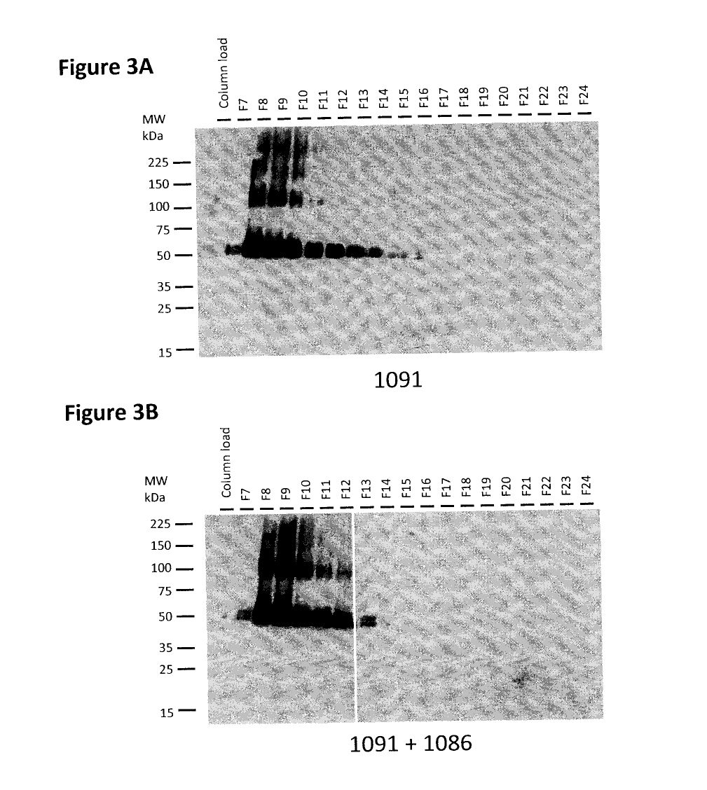

FIGS. 3A and 3B, show an example of an elution profile of a size exclusion chromatography analysis of a plant extract comprising VLPs. In this case, VLPs comprising native rabies virus structural proteins elute in fractions 8 to approx. 11, rosettes and high molecular weight structures elute from about fractions 12 to about 14, and lower molecular weight, or soluble form of the native rabies virus structural proteins elute in fractions from about 15 to about 17.

The VLPs may be purified or extracted using any suitable method for example chemical or biochemical extraction. VLPs are relatively sensitive to desiccation, heat, pH, surfactants and detergents. Therefore it may be useful to use methods that maximize yields, minimize contamination of the VLP fraction with cellular proteins, maintain the integrity of the proteins, or VLPs, and, where required, the associated lipid envelope or membrane, methods of loosening the cell wall to release the proteins, or VLP. For example, methods that produce protoplast and/or spheroplasts may be used (see for example WO 2011/035422, which is incorporated herein by reference) to obtain VLPs as described herein. Minimizing or eliminating the use of detergence or surfactants such for example SDS or Triton X-100 may be beneficial for improving the yield of VLP extraction. VLPs may be then assessed for structure and size by, for example, electron microscopy, or by size exclusion chromatography as mentioned above.

The one or more than one or more genetic constructs of the present invention may be expressed in any suitable plant host that is transformed by the nucleotide sequence, or constructs, or vectors of the present invention. Examples of suitable hosts include, but are not limited to, agricultural crops including alfalfa, canola, Brassica spp., maize, Nicotiana spp., alfalfa, potato, ginseng, pea, oat, rice, soybean, wheat, barley, sunflower, cotton and the like.

The one or more genetic constructs of the present invention can further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed nontranslated regions containing a polyadenylation signal of Agrobacterium tumor inducing (Ti) plasmid genes, such as the nopaline synthase (NOS) gene, plant genes such as the soybean storage protein genes, the small subunit of the ribulose-1,5-bisphosphate carboxylase gene (ssRUBISCO; U.S. Pat. No. 4,962,028; which is incorporated herein by reference), the promoter used in regulating plastocyanin expression, described in U.S. Pat. No. 7,125,978 (which is incorporated herein by reference).

One or more of the genetic constructs of the present invention may also include further enhancers, either translation or transcription enhancers, as may be required. Enhancers may be located 5′ or 3′ to the sequence being transcribed. Enhancer regions are well known to persons skilled in the art, and may include an ATG initiation codon, adjacent sequences or the like. The initiation codon, if present, may be in phase with the reading frame (“in frame”) of the coding sequence to provide for correct translation of the transcribed sequence.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (Gene 100: 247-250 (1991), Scheid et al. (Mol. Gen. Genet. 228: 104-112, 1991), Guerche et al. (Plant Science 52: 111-116, 1987), Neuhause et al. (Theor. Appl Genet. 75: 30-36, 1987), Klein et al., Nature 327: 70-73 (1987); Howell et al. (Science 208: 1265, 1980), Horsch et al. (Science 227: 1229-1231, 1985), DeBlock et al., Plant Physiology 91: 694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J Virol Meth, 105:343-348, 2002,), U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

As described below, transient expression methods may be used to express the constructs of the present invention (see Liu and Lomonossoff, 2002, Journal of Virological Methods, 105:343-348; which is incorporated herein by reference). Alternatively, a vacuum-based transient expression method, as described by Kapila et al., 1997, which is incorporated herein by reference) may be used. These methods may include, for example, but are not limited to, a method of Agro-inoculation or Agro-infiltration, syringe infiltration, however, other transient methods may also be used as noted above. With Agro-inoculation, Agro-infiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

To aid in identification of transformed plant cells, the constructs of this invention may be further manipulated to include plant selectable markers. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used.

Also considered part of this invention are transgenic plants, plant cells or seeds containing the gene construct of the present invention. Methods of regenerating whole plants from plant cells are also known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue cultures.

The present invention includes nucleotide sequences:

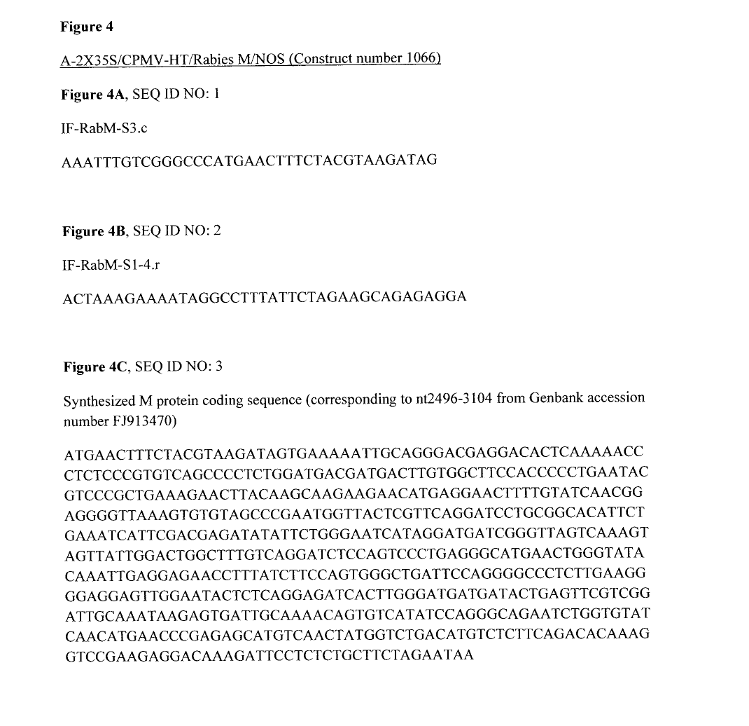

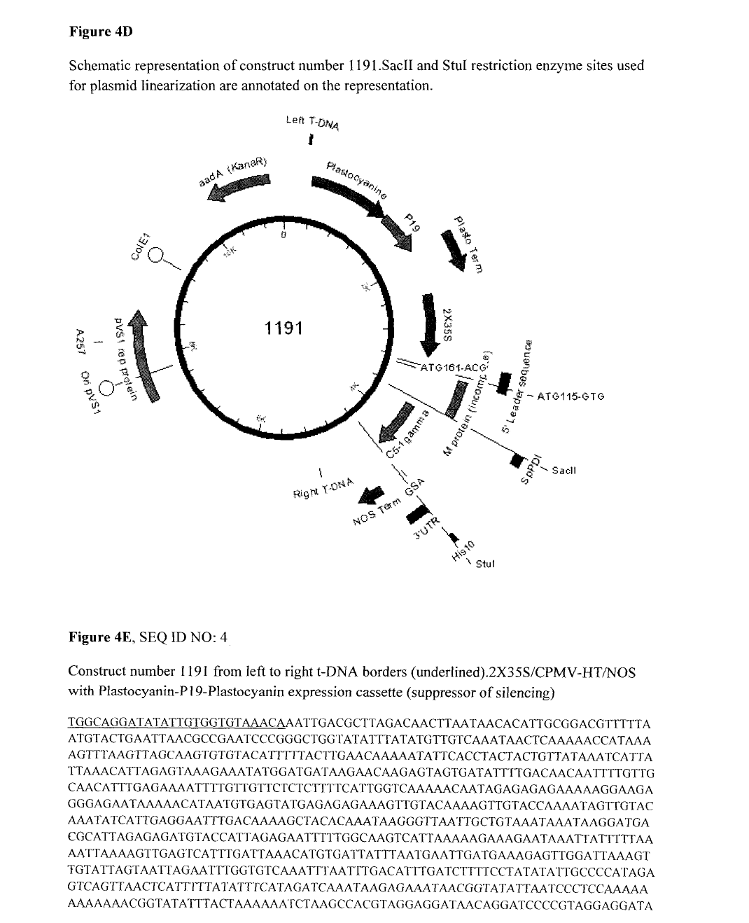

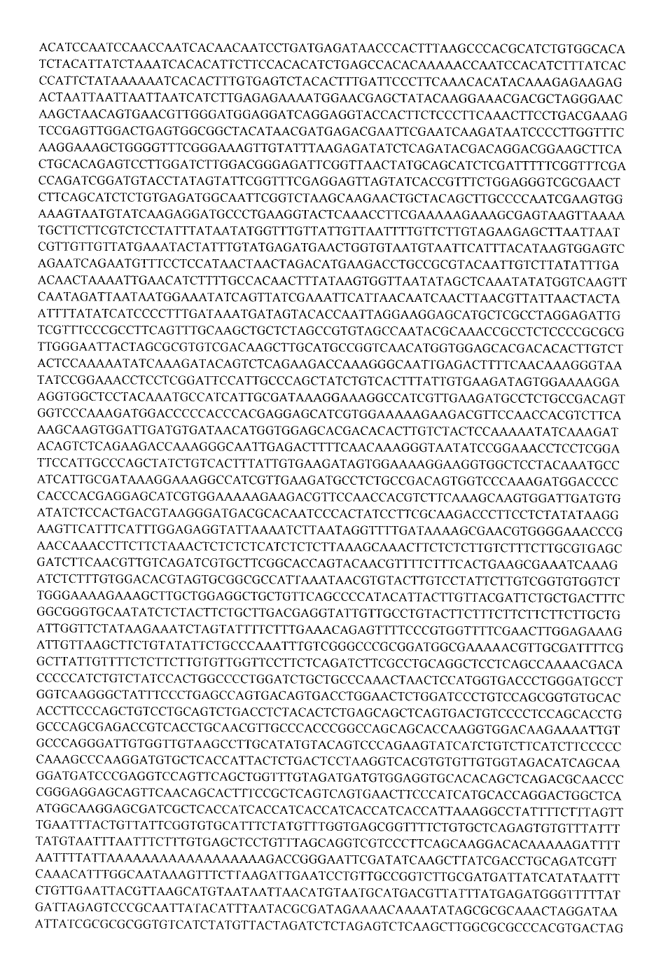

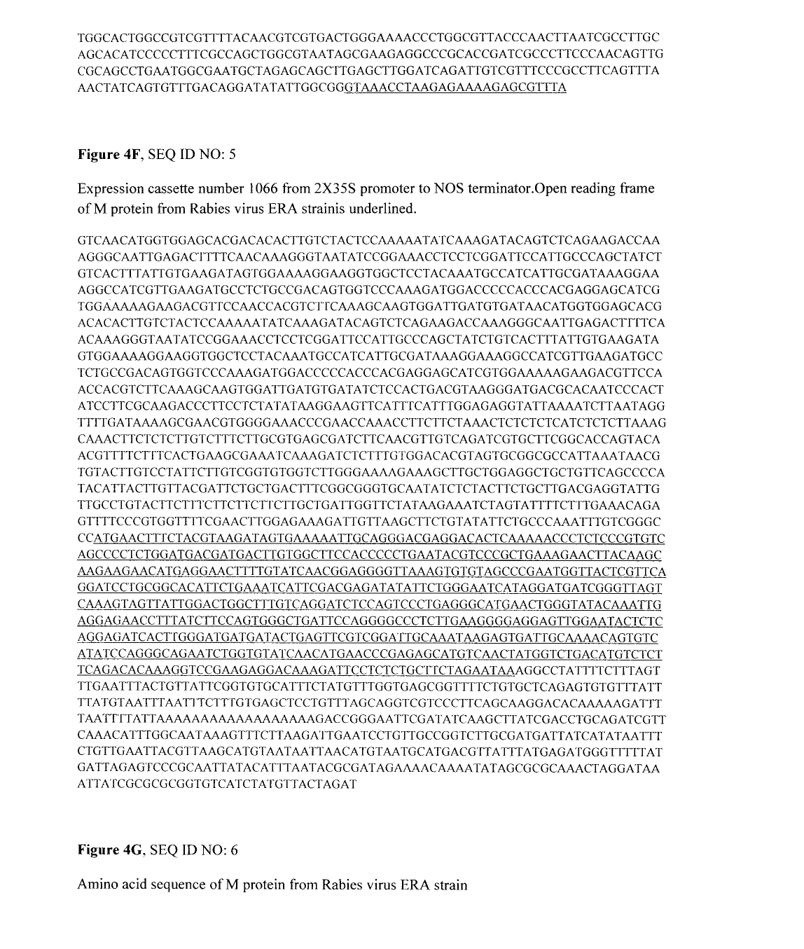

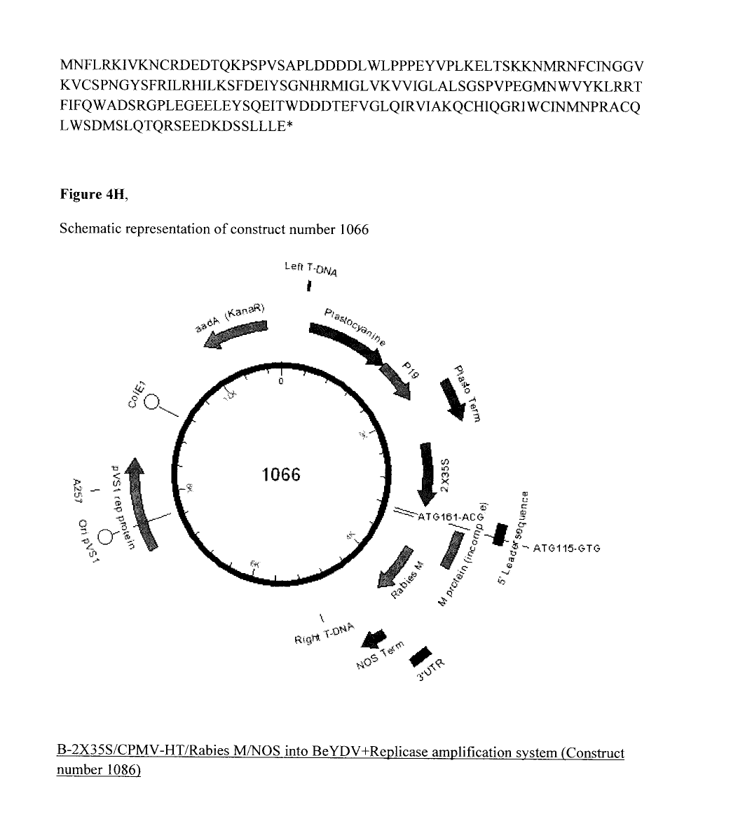

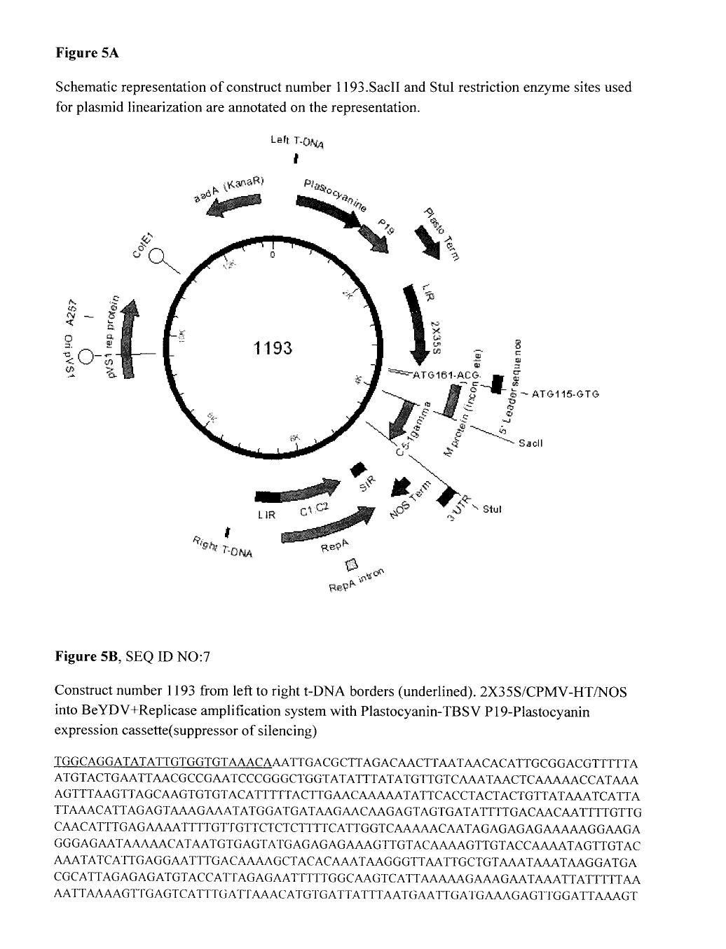

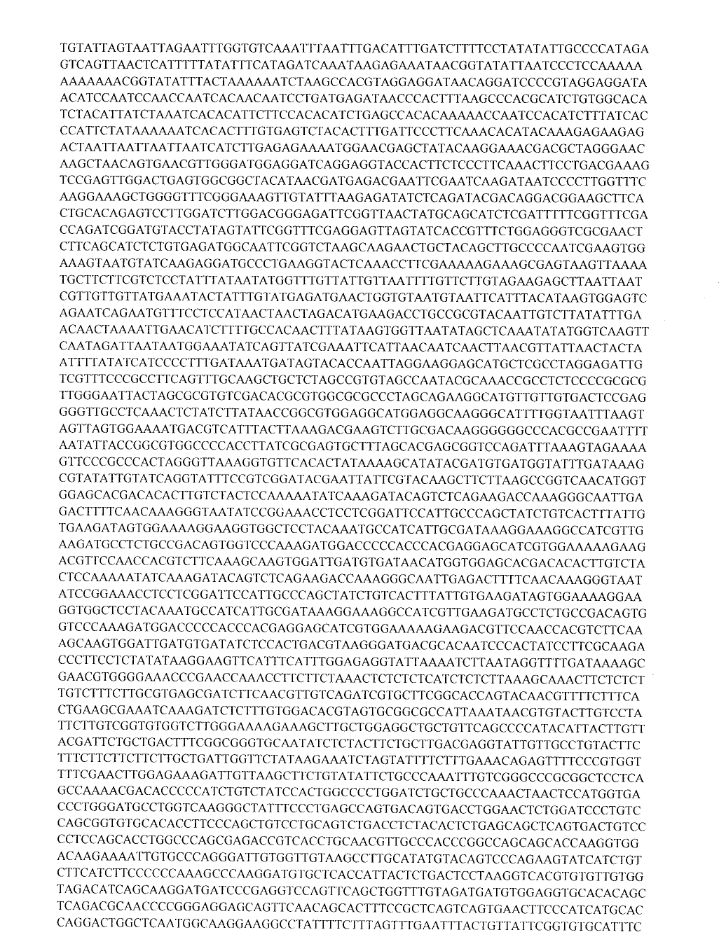

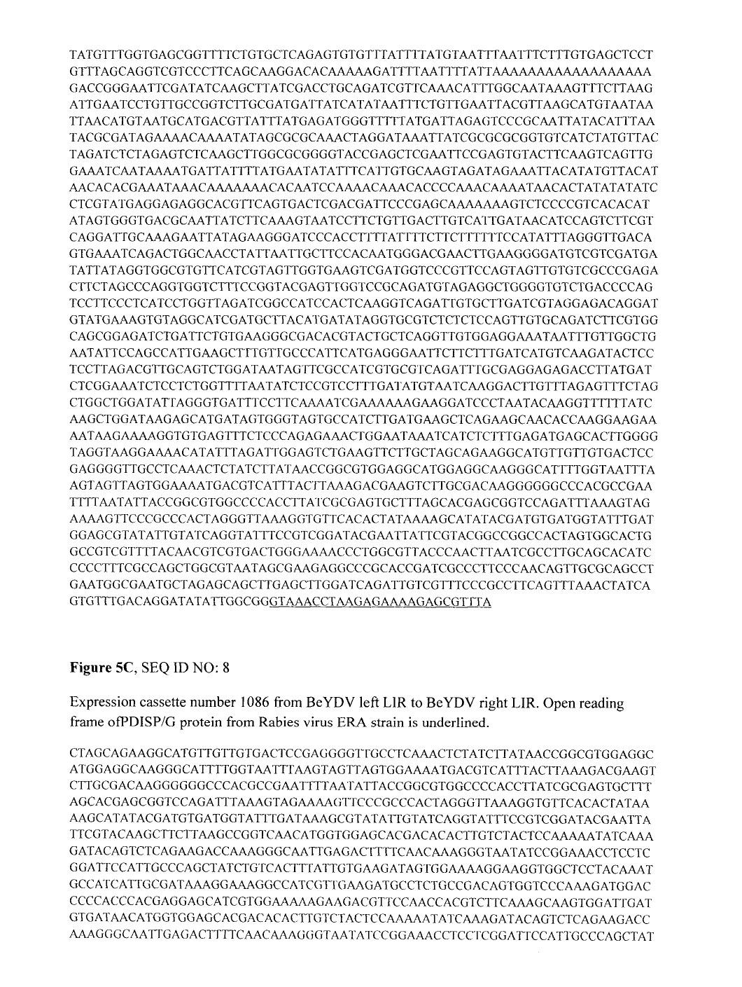

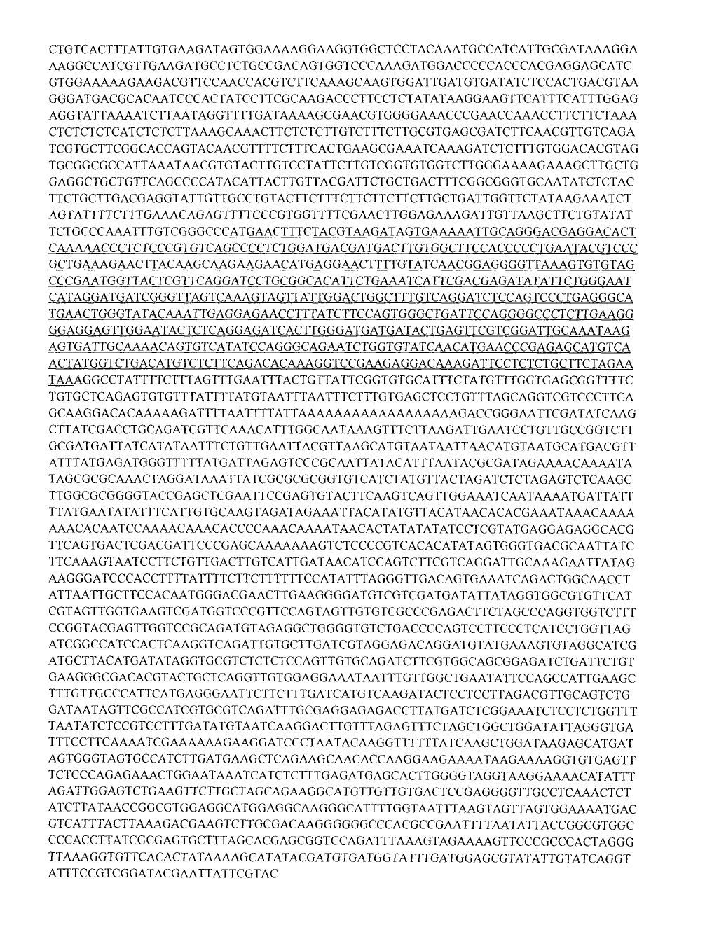

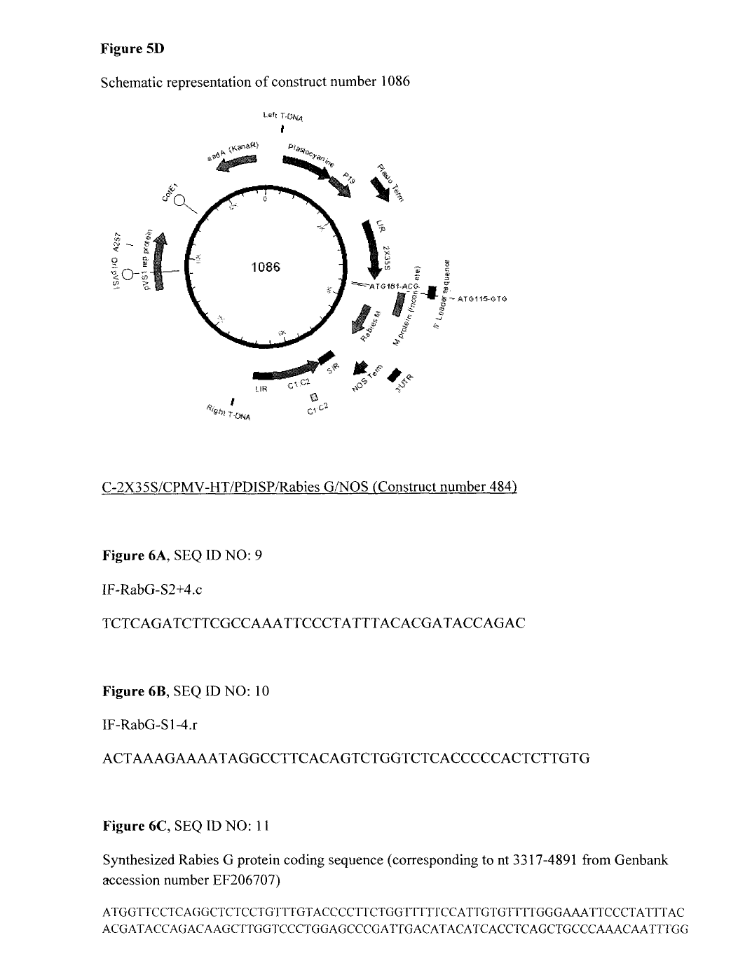

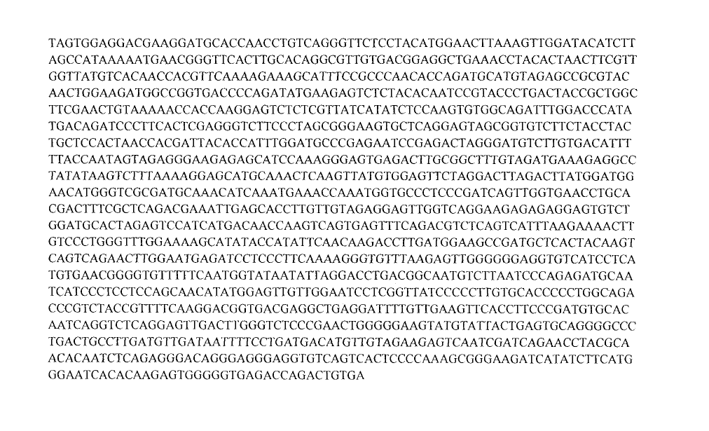

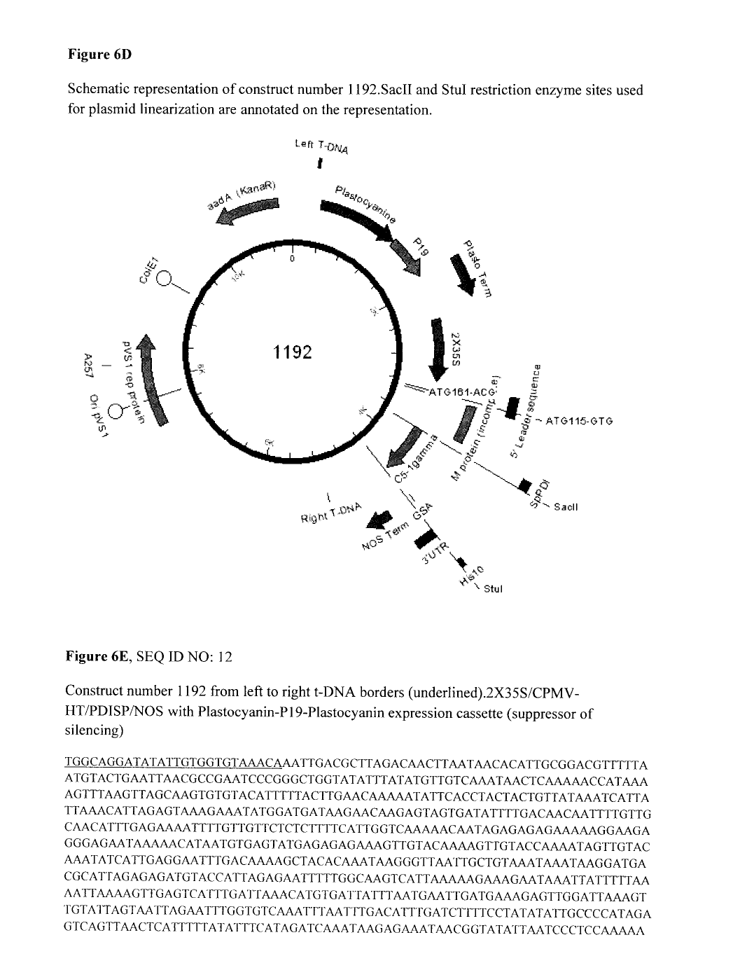

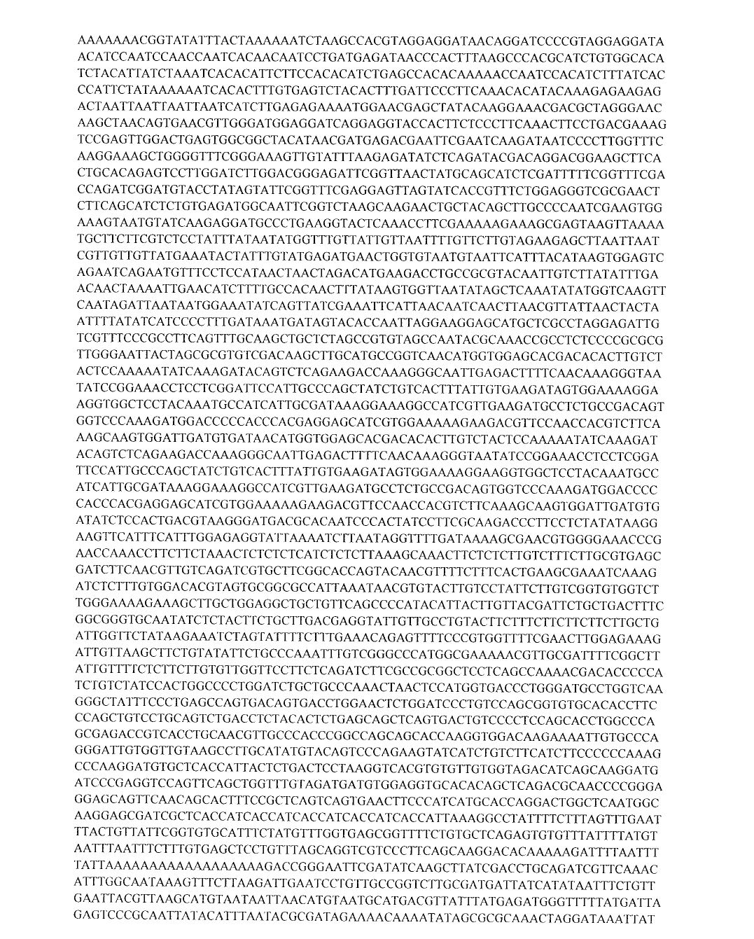

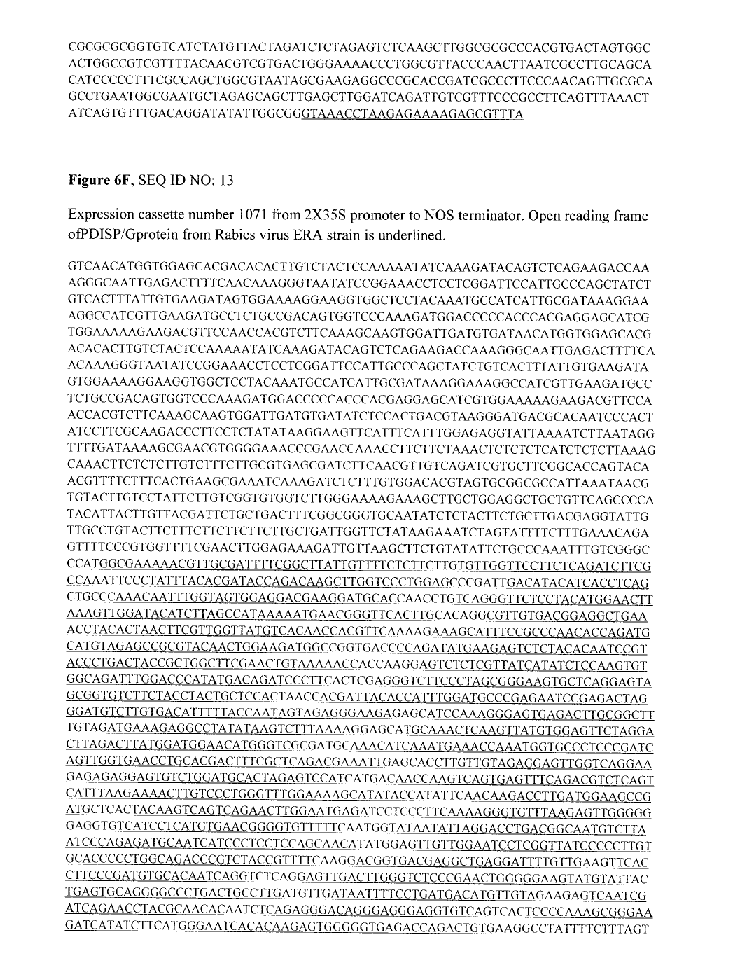

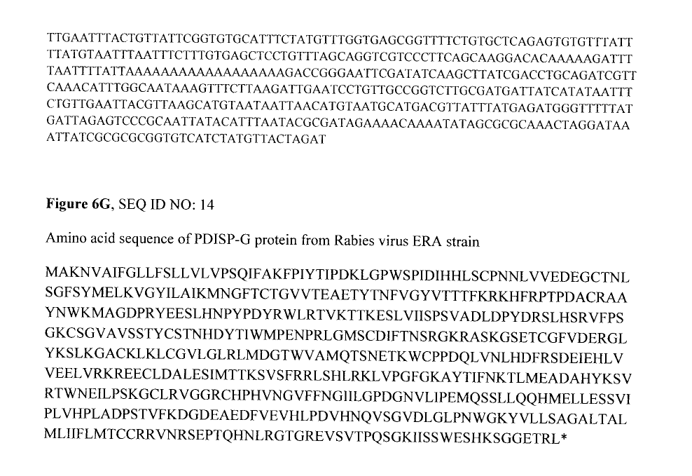

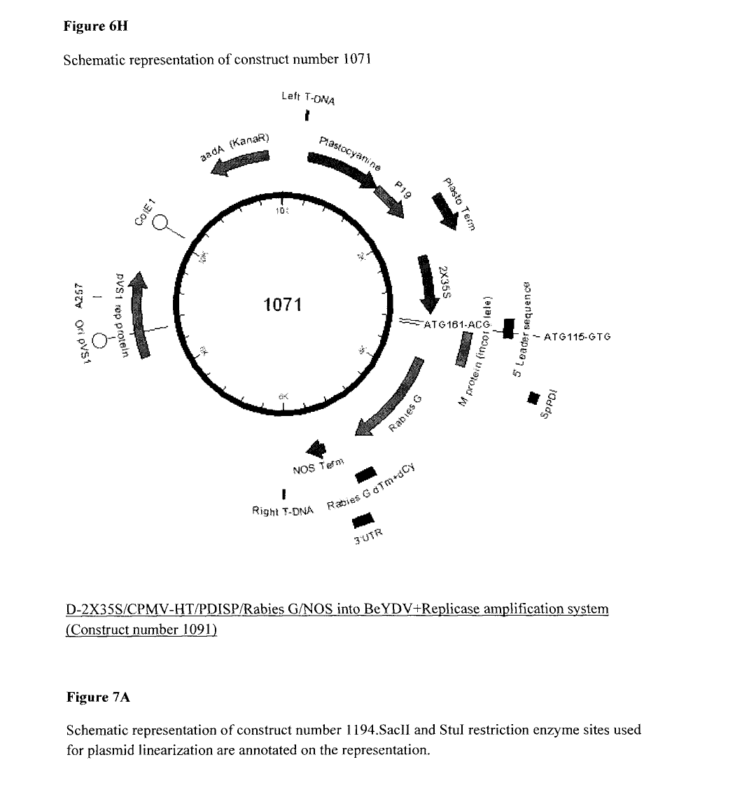

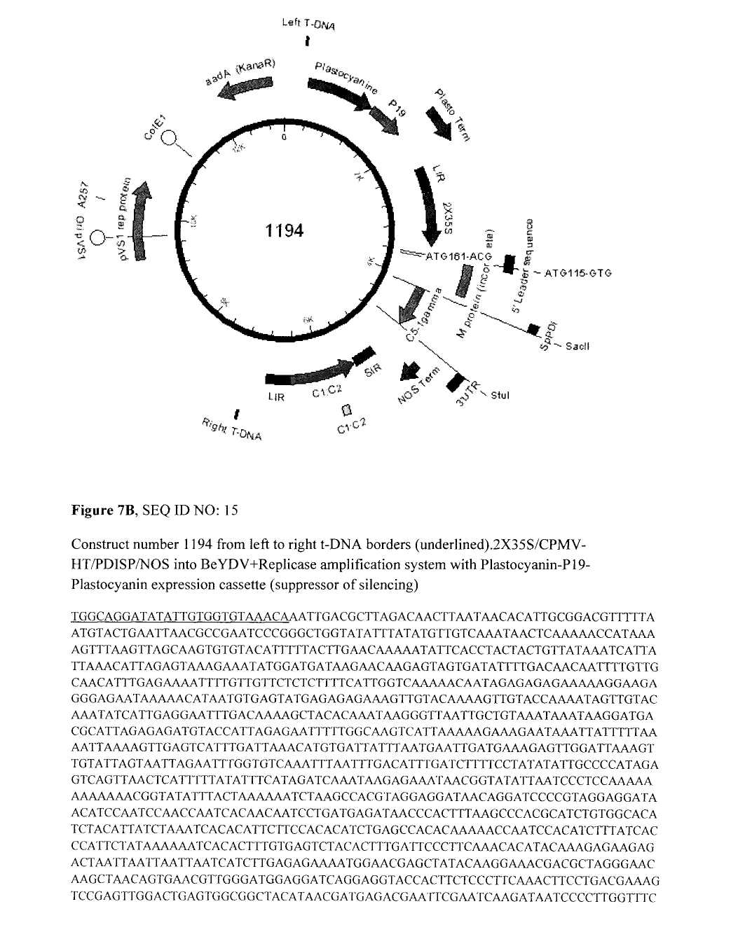

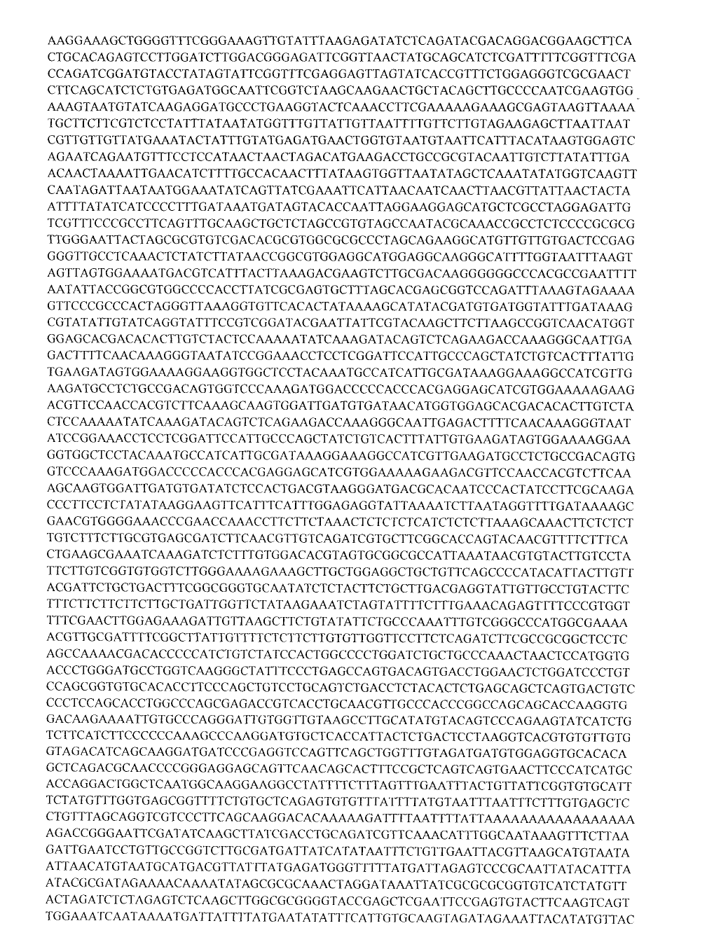

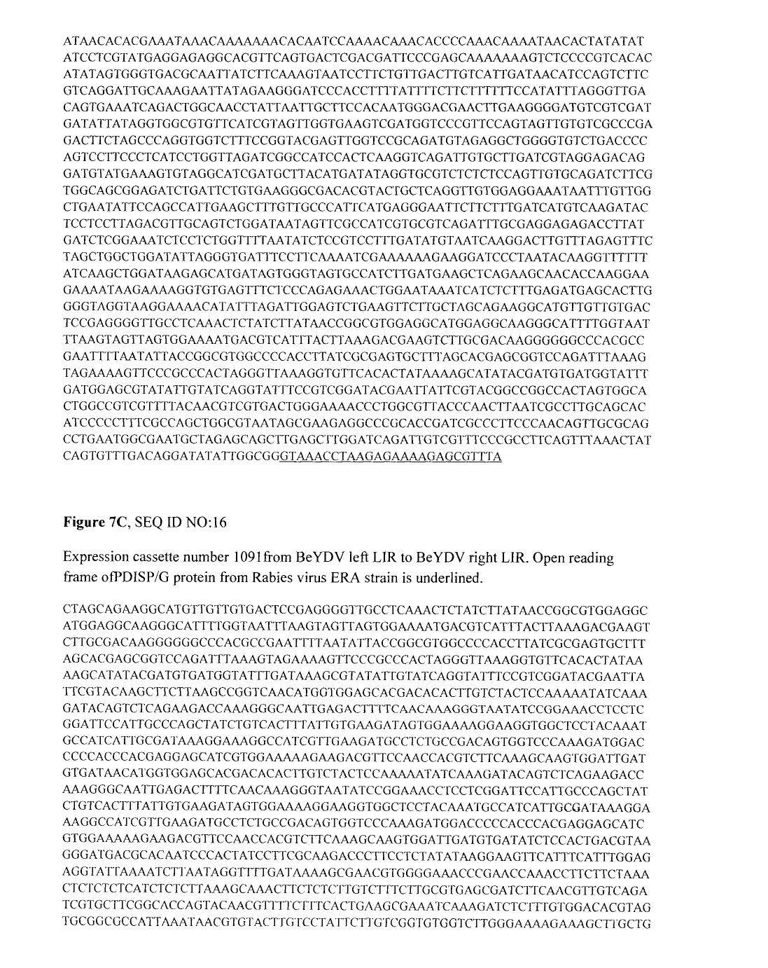

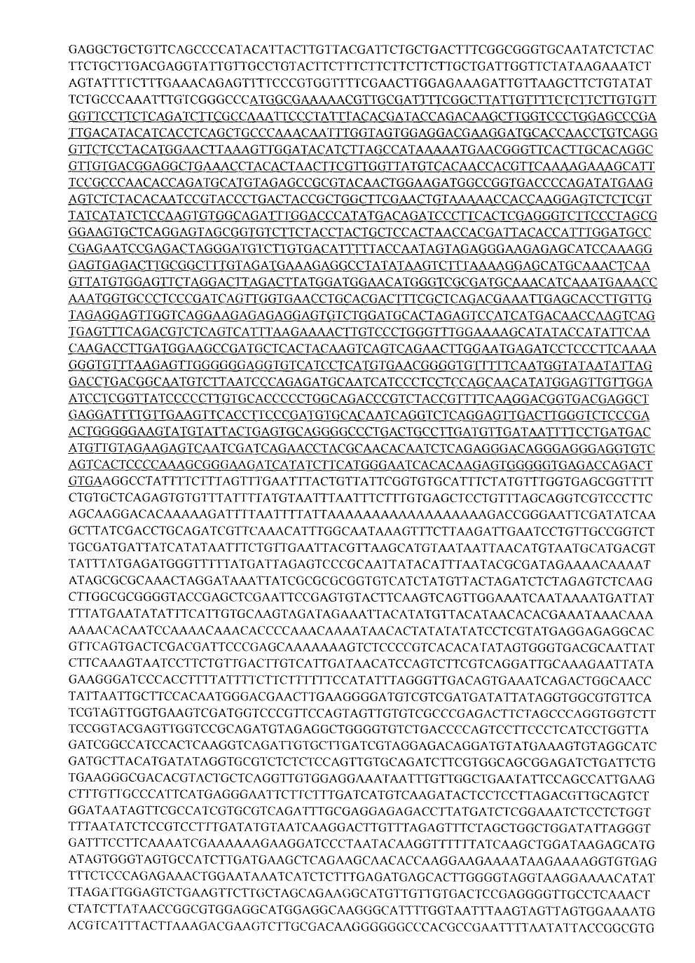

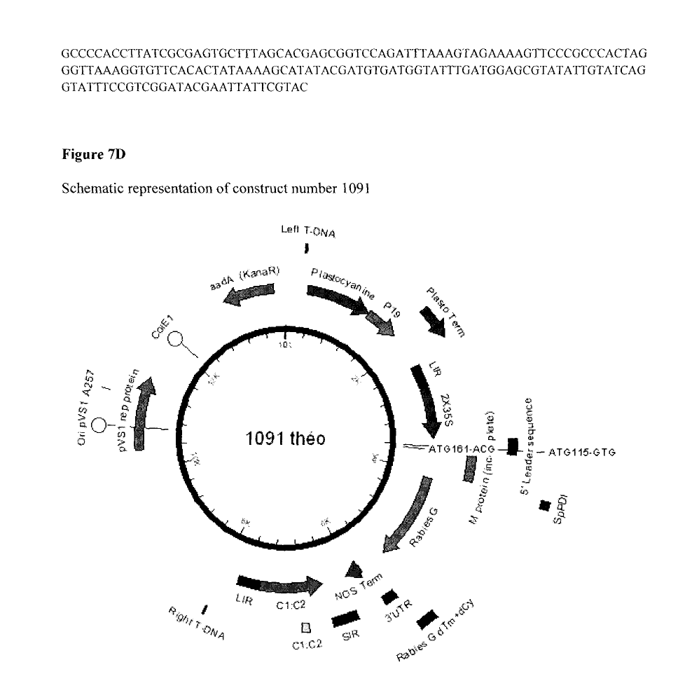

TABLE 1
List of Sequence Identification numbers.

| SEQ ID | | Table/ |
|---|---|---|
| NO: | Description | Figure |
| 1 | IF-RabM-S3.c | FIG. 4A |
| 2 | IF-RabM-S1-4.r | FIG. 4B |
| 3 | Synthesized M protein coding sequence | FIG. 4C |
| | (corresponding to nt2496-3104 from Genbank | |
| | accession number FJ913470) | |
| 4 | Construct number 1191 from left to right t-DNA | FIG. 4E |
| | borders (underlined).2X35S/CPMV-HT/NOS with | |
| | Plastocyanin-P19-Plastocyanin expression | |
| | cassette (suppressor of silencing) | |
| 5 | Expression cassette number 1066 from 2X35S | FIG. 4F |
| | promoter to NOS terminator. Open reading frame | |
| | of M protein from Rabies virus ERA strainis | |
| | underlined. | |
| 6 | Amino acid sequence of M protein from Rabies | FIG. 4G |
| | virus ERA strain | |
| 7 | Construct number 1193 from left to right t-DNA | FIG. 5B |
| | borders (underlined). 2X35S/CPMV-HT/NOS into | |
| | BeYDV + Replicase amplification system with | |
| | Plastocyanin-TBSV P19-Plastocyanin expression | |
| | cassette(suppressor of silencing) | |
| 8 | Expression cassette number 1086 from BeYDV left | FIG. 5C |
| | LIR to BeYDV right LIR. Open reading frame | |
| | ofPDISP/G protein from Rabies virus ERA strain | |
| | is underlined. | |
| 9 | IF-RabG-S2 + 4.c | FIG. 6A |
| 10 | IF-RabG-S1 − 4.r | FIG. 6B |
| 11 | Synthesized Rabies G protein coding sequence | FIG. 6C |
| | (corresponding to nt 3317-4891 from Genbank | |
| | accession number EF206707) | |
| 12 | Construct number 1192 from left to right t-DNA | FIG. 6E |
| | borders (underlined).2X35S/CPMV-HT/PDISP/ | |
| | NOS with Plastocyanin-P19-Plastocyanin expression | |
| | cassette (suppressor of silencing) | |
| 13 | Expression cassette number 1071 from 2X35S | FIG. 6F |
| | promoter to NOS terminator. Open reading frame | |
| | ofPDISP/Gprotein from Rabies virus ERA strain | |
| | is underlined. | |
| 14 | Amino acid sequence of PDISP-G protein from | FIG. 6G |
| | Rabies virus ERA strain | |
| 15 | Construct number 1194 from left to right t-DNA | FIG. 7B |
| | borders (underlined).2X35S/CPMV- HT/PDISP/ | |
| | NOS into BeYDV + Replicase amplification system | |
| | with Plastocyanin-P19-Plastocyanine | |
| | expression cassette (suppressor of silencing) | |
| 16 | Expression cassette number 1091from BeYDV | FIG. 7C |
| | left LIR to BeYDV right LIR. Open reading | |
| | frame ofPDISP/G protein from Rabies virus | |
| | ERA strain is underlined. | |

The present invention will be further illustrated in the following examples.

### EXAMPLES

### Constructs

TABLE 2
Constructs comprising sequences encoding native rabies virus
structural protein

| Expression | Amplification | Signal | Structural | Construct |
|---|---|---|---|---|
| system | system | peptide | Protein | number |
| CPMV HT | — | — | Rabies M | 1066 |
| CPMV HT | BeYDV + rep | — | Rabies M | 1086 |
| CPMV HT | — | Sp PDI | Rabies G | 1071 |
| CPMV HT | BeYDV + rep | Sp PDI | Rabies G | 1091 |

### Example 1

### Assembly of Expression Cassettes with Rabies Protein

### A-2×35S/CPMV-HT/Rabies M/NOS (Construct Number 1066)

A sequence encoding M protein from Rabies virus ERA strain was cloned into 2×35S/CPMV-HT/NOS expression system in a plasmid containing Plasto_pro/P19/Plasto_terexpression cassette using the following PCR-based method. A fragment containing the complete M protein coding sequence was amplified using primers IF-RabM-S3.c (FIG. 4A, SEQ ID NO:1) and IF-RabM-S1−4.r (FIG. 4B, SEQ ID NO: 2) using synthesized M gene (corresponding to nt 2496-3104 from Genbank accession number FJ913470) (FIG. 4C, SEQ ID NO: 3) as template. The PCR product was cloned in 2×35S/CPMV-HT/NOS expression system using In-Fusion cloning system (Clontech, Mountain View, Calif.). Construct 1191 (FIG. 4D) was digested with SacII and StuI restriction enzyme and the linearized plasmid was used for the In-Fusion assembly reaction. Construct number 1191 is an acceptor plasmid intended for “In Fusion” cloning of genes of interest in a CPMV-HT-based expression cassette. It also incorporates a gene construct for the co-expression of the TBSV P19 suppressor of silencing under the alfalfa Plastocyanin gene promoter and terminator. The backbone of construct number 1191 is a pCAMBIA binary plasmid and the sequence from left to right t-DNA borders is presented in FIG. 4E (SEQ ID NO:4). The resulting construct was given number 1066 (FIG. 4F, SEQ ID NO: 5). The amino acid sequence of M protein from Rabies virus ERA strain is presented in FIG. 4G (SEQ ID NO: 6). A representation of plasmid 1066 is presented in FIG. 4H.

B-2×35S/CPMV-HT/Rabies M/NOS intoBeYDV+Replicase Amplification System (Construct Number 1086)

A sequence encoding M protein from Rabies virus ERA strain was cloned into 2×35S/CPMV-HT/NOS expression system comprising the BeYDV+replicase amplification system in a plasmid containing Plasto_pro/P19/Plasto_ter expression cassette using the following PCR-based method. A fragment containing the complete M protein coding sequence was amplified using primers IF-RabM-S3.c (FIG. 4A, SEQ ID NO: 1) and IF-RabM-S1−4.r (FIG. 4B, SEQ ID NO: 2) using synthesized M gene (corresponding to nt 2496-3104 from Genbank accession number FJ913470) (FIG. 4C, SEQ ID NO: 3) as template. The PCR product was cloned in 2×35S/CPMV-HT/NOS expression system using In-Fusion cloning system (Clontech, Mountain View, Calif.). Construct 1193 (FIG. 5A, SEQ ID NO: B1) was digested with SacII and StuI restriction enzyme and the linearized plasmid was used for the In-Fusion assembly reaction. Construct number 1193 is an acceptor plasmid intended for “In Fusion” cloning of genes of interest in a CPMV-HT-based expression cassette into the BeYDV amplification system. It also incorporates a gene construct for the co-expression of the TBSV P19 suppressor of silencing under the alfalfa Plastocyanin gene promoter and terminator. The backbone of construct number 1193 is a pCAMBIA binary plasmid and the sequence from left to right t-DNA borders is presented in FIG. 5B (SEQ ID NO: 7). The resulting construct was given number 1086 (FIG. 5C, SEQ ID NO: 8). The amino acid sequence of M protein from Rabies virus ERA strain is presented in FIG. 4G (SEQ ID NO: 6). A representation of plasmid 1086 is presented in FIG. 5D.

### C-2×35S/CPMV-HT/PDISP/Rabies G/NOS (Construct Number 1071)

A sequence encoding G protein from Rabies virus ERA strain was cloned into 2×35S-CPMV-HT-PDISP-NOS expression system in a plasmid containing Plasto_pro/P19/Plasto_ter expression cassette using the following PCR-based method. A fragment containing the G protein coding sequence without its wild type signal peptide was amplified using primersIF-RabG-S2+4.c (FIG. 6A, SEQ ID NO:9) and IF-RabG-S1−4.r (FIG. 6B, SEQ ID NO: 10), using synthesized G gene (Corresponding to nt 3317-4891 from Genbank accession number EF206707) (FIG. 6C, SEQ ID NO: 11) as template. The PCR product was cloned in-frame with alfalfa PDI signal peptide in 2×35S/CPMV-HT/NOS expression system using In-Fusion cloning system (Clontech, Mountain View, Calif.). Construct 1192 (FIG. 6D) was digested with SacII and StuI restriction enzyme and the linearized plasmid was used for the In-Fusion assembly reaction. Construct number 1192 is an acceptor plasmid intended for “In Fusion” cloning of genes of interest in frame with an alfalfa PDI signal peptide in a CPMV-HT-based expression cassette. It also incorporates a gene construct for the co-expression of the TBSV P19 suppressor of silencing under the alfalfa Plastocyanin gene promoter and terminator. The backbone of construct 1192 is a pCAMBIA binary plasmid and the sequence from left to right t-DNA borders is presented in FIG. 6E (SEQ ID NO: 12). The resulting construct was given number 1071 (FIG. 6F, SEQ ID NO: 13). The amino acid sequence of PDISP/G protein from Rabies virus ERA strain is presented in FIG. 6G (SEQ ID NO: 14). A representation of plasmid 1071 is presented in FIG. 6H.

### D-2×35S/CPMV-HT/PDISP/Rabies G/NOSintoBeYDV+Replicase Amplification System (Construct Number 1091)

A sequence encoding G protein from Rabies virus ERA strain was cloned into 2×35S/CPMV-HT/PDISP/NOS comprising the BeYDV+replicase amplification system in a plasmid containing Plasto_pro/P19/Plasto_ter expression cassette using the following PCR-based method. A fragment containing Rabies G protein coding sequence without his wild type signal peptide was amplified using primers IF-RabG-S2+4.c (FIG. 6A, SEQ ID NO: 9) and IF-RabG-S1−4.r (FIG. 6B, SEQ ID NO: 10), using synthesized G gene (Corresponding to nt 3317-4891 from Genbank accession number EF206707) (FIG. 6C, SEQ ID NO: 11) as template. The PCR product was cloned in-frame with alfalfa PDI signal peptide in 2×35S/CPMV-HT/NOS expression cassette into the BeYDV amplification system using In-Fusion cloning system (Clontech, Mountain View, Calif.). Construct number 1194 (FIG. 7A) was digested with SacII and StuI restriction enzyme and the linearized plasmid was used for the In-Fusion assembly reaction. Construct number 1194 is an acceptor plasmid intended for “In Fusion” cloning of genes of interest in frame with an alfalfa PDI signal peptide in a CPMV-HT-based expression cassette into the BeYDV amplification system. It also incorporates a gene construct for the co-expression of the TBSV P19 suppressor of silencing under the alfalfa Plastocyanin gene promoter and terminator. The backbone of construct number 1194 is a pCAMBIA binary plasmid and the sequence from left to right t-DNA borders is presented in FIG. 7B (SEQ ID NO: 15). The resulting construct was given number 1091 (FIG. 7C, SEQ ID NO: 16). The amino acid sequence of Influenza PDISP/G protein from Rabies virus ERA strain is presented in FIG. 6G (SEQ ID NO: 14). A representation of plasmid 1091 is presented in FIG. 7D.

### Example 2

### Preparation of Plant Biomass, Inoculum and Agro Infiltration

The terms “biomass” and “plant matter” as used herein are meant to reflect any material derived from a plant. Biomass or plant matter may comprise an entire plant, tissue, cells, or any fraction thereof. Further, biomass or plant matter may comprise intracellular plant components, extracellular plant components, liquid or solid extracts of plants, or a combination thereof. Further, biomass or plant matter may comprise plants, plant cells, tissue, a liquid extract, or a combination thereof, from plant leaves, stems, fruit, roots or a combination thereof. A portion of a plant may comprise plant matter or biomass.

Nicotiana benthamiana plants were grown from seeds in flats filled with a commercial peat moss substrate. The plants were allowed to grow in the greenhouse under a 16/8 photoperiod and a temperature regime of 25° C. day/20° C. night. Three weeks after seeding, individual plantlets were picked out, transplanted in pots and left to grow in the greenhouse for three additional weeks under the same environmental conditions.

Agrobacteria transfected with each construct were grown in a YEB medium supplemented with 10 mM 2-(N-morpholino)ethanesulfonic acid (MES), 20 μM acetosyringone, 50 μg/mlkanamycin and 25 μg/ml of carbenicillin pH 5.6 until they reached an OD₆₀₀ between 0.6 and 1.6. Agrobacterium suspensions were centrifuged before use and resuspended in infiltration medium (10 mM MgCl₂ and 10 mM MES pH 5.6) and stored overnight at 4° C. On the day of infiltration, culture batches were diluted in 2.5 culture volumes and allowed to warm before use. Whole plants of N. benthamiana were placed upside down in the bacterial suspension in an air-tight stainless steel tank under a vacuum of 20-40 Torr for 2-min. Plants were returned to the greenhouse for a 2-6 day incubation period until harvest.

A. tumefaciens strains comprising the various constructs as described herein are referred to by using an “AGL1” prefix. For example A. tumefaciens comprising construct number 1091 is termed “AGL1/1091”.

### Leaf Harvest and Total Protein Extraction (Mechanical Extraction)

Following incubation, the aerial part of plants was harvested, frozen at −80° C. and crushed into pieces. Total soluble proteins were extracted by homogenizing (Polytron) each sample of frozen-crushed plant material in 3 volumes of cold 50 mM Tris pH 8.0, 150 mM NaCl, 0.1% Triton X-100 and 1 mM phenylmethanesulfonyl fluoride. After homogenization, the slurries were centrifuged at 10,000 g for 10 min at 4° C. and these clarified crude extracts (supernatant) kept for analysis.

### Leaf Harvest and Total Protein Extraction (Biochemical Extraction)

Following incubation, the aerial part of plants was harvested and cut into small pieces (3 mm square) with a rolling cutter and special care was taken not to crush the leaves. Cut biomass was incubated 15-17 hours in 2.5 volumes of 200 mM mannitol, 125 mM citrate, 75 mM NaPO₄, 500 mM NaCl, 25 mM ethylenediaminetetraacetic acid, 1% (v/v) Multifect CX CG (Genencor, Cat. No. A03140G190), 1% (v/v) Multifect CX B (Genencor, Cat. No. A03042G190) and 1% (v/v) Multifect Pectinase FE (Genencor, Cat. No. A02080G190) pH 6.5 at 21° C. under agitation at 100-200 rpm. Digested biomass was then filtered on Miracloth (Calbiochem, Cat 475855) and centrifuged at 10,000 g for 10 min at 4° C. The supernatant was transferred to a clean tube and centrifuged again under the same conditions and the supernatant was kept for analysis.

### Protein Analysis and Immunoblotting

The total protein content of clarified crude extracts was determined by the Bradford assay (Bio-Rad, Hercules, Calif.) using bovine serum albumin as the reference standard. Proteins were separated by SDS-PAGE and electrotransferred onto polyvinylene difluoride (PVDF) membranes (Roche Diagnostics Corporation, Indianapolis, Ind.) for immunodetection. Prior to immunoblotting, the membranes were blocked with 5% skim milk and 0.1% Tween-20 in Tris-buffered saline (TBS-T) for 16-18 h at 4° C. Immunoblotting was performed by incubation with 0.25 μg/μl of a Santa Cruz SC-57995 primary antibody in 2% skim milk in TBS-Tween 20 0.1%. Chemiluminescence detection was carried on after incubation with peroxidase-conjugated goat anti mouse, (JIR, 115-035-146) secondary antibody diluted 1:10,000 in 2% skim milk in TBS-Tween 20 0.1%. Immunoreactive complexes were detected by chemiluminescence using luminol as the substrate (Roche Diagnostics Corporation).

### Size Exclusion Chromatography of Protein Extract

For size exclusion chromatography (SEC), supernatant from biochemically extracted biomass was concentrated by centrifugation at 70,000 g for 20 min at 4° C. and the resulting pellet was resuspended in 1/50 volume of cold 50 mM Tris pH 8.0, 150 mM NaCl. Size exclusion chromatography columns of 32 ml Sephacryl™ S-500 high resolution beads (S-500 HR: GE Healthcare, Uppsala, Sweden, Cat. No. 17-0613-10) were packed and equilibrated with equilibration/elution buffer (50 mM Tris pH8, 150 mM NaCl). One and a half millilitre of concentrated extract was loaded onto the column followed by an elution step with 45 mL of equilibration/elution buffer. The elution was collected in fractions of 1.5 mL relative protein content of eluted fractions was monitored by mixing 10 μL of the fraction with 200 μL of diluted Bio-Rad protein dye reagent (Bio-Rad, Hercules, Calif.). Two hundred microliters from each fraction were precipitated by addition of 5 volumes of ice cold acetone followed by freezing overnight at −20° C. Precipitated proteins were pelleted by centrifugation at 20000 g for 10 min. (4° C.) and resuspended in 50 μl of hot SDS-PAGE sample loading buffer. The column was washed with 2 column volumes of 0.2N NaOH followed by 10 column volumes of 50 mM Tris pH8, 150 mM NaCl, 20% ethanol. Each separation was followed by a calibration of the column with Blue Dextran 2000 (GE Healthcare Bio-Science Corp., Piscataway, N.J., USA). Elution profiles of Blue Dextran 2000 and host soluble proteins were compared between each separation to ensure uniformity of the elution profiles between the columns used.

### Example 3

### Evaluation of Rabies G Protein Expression and Co-Expression Strategies

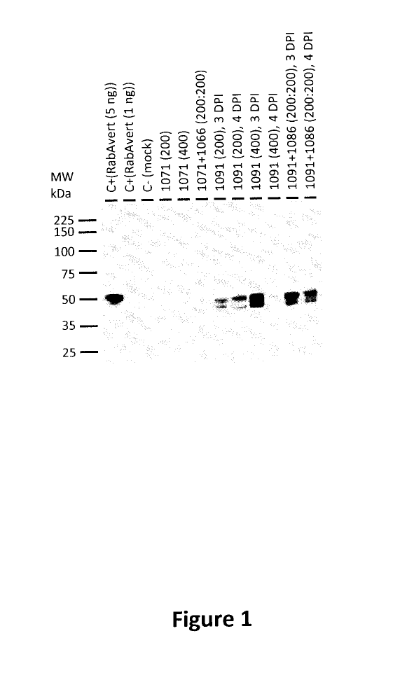

Nicotiana benthamiana plants were agro-infiltrated with AGL1/1071 (with or without AGL1/1066) or AGL1/1091 (with or without AGL1/1086) inoculums at different concentration and leaves were harvested after 5 days post infiltration (DPI) for 1071− and 1071+1066-infiltrated plants or 3 to 4 DPI for 1091− and 1091+1086-infiltrated plants. Western blot analysis of leaf protein extracts from transformed plants showed that the BeYDV elements were required to reach a detectable G protein accumulation level (compare 1071- and 1091-infiltrated plants in FIG. 1). Maximum accumulation level was reached at 3 DPI for plants infiltrated with AGL1/1091 with or without co-expression of M protein (AGL1/1086) (FIG. 1).

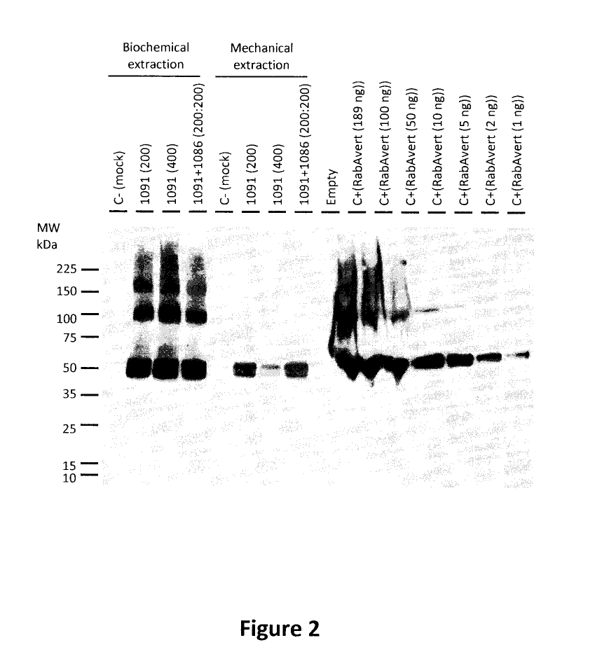

The biochemical extraction method was compared to the mechanical extraction for its capacity to release rabies G protein from the leaves of transformed plants. Western blot analysis of extracts obtained by biochemical extraction and mechanical extraction on 1091− and 1091+1086-infiltrated plants showed that the protein extracts from biochemical extraction contained significantly more G protein than the extract from mechanical extraction (FIG. 2).

Protein extracts from 1091− and 1091+1086-infiltrated plants were subjected to size exclusion chromatography to assess their assembly into high molecular weight structures. Elution fractions were collected and an aliquot of each fraction was concentrated by acetone precipitation and analyzed for protein G content by western blot. As shown in FIG. 3A, protein G content peaked in SEC elution fractions 8 to 10, corresponding to the void volume of the column where enveloped VLPs are expected to be found. A similar elution profile was obtained when separating, by SEC, protein extracts from plants expressing rabies protein G with protein M (1091+1086-infiltrated plants, FIG. 3B).

### Example 4

### Animal Studies

Rapid Purification of Rab-VLP from Plants

N. benthamiana plants were agroinfiltrated with AGL1/1091 as described for example in WO/2011/035422 which is incorporated herein by reference. Extraction of Rab-VLP (also referred to as NG-VLP, Native G protein VLP, G-VLP or G protein VLP) was undertaken as described above. Briefly, leaves were collected on day 4 post-infiltration, cut into ˜1 cm² pieces and digested for 15 h at room temperature in an orbital shaker. The digestion buffer contained 1.0% (v/v) Multifect Pectinase FE, 1.0% (v/v) Multifect CX CG and/or 1.0% (v/v) Multifect CX B (all from Genencor), each in a solution of 200 mM Mannitol, 75 mM Citrate, 0.04%, 500 mM NaCl sodium bisulfate pH 6.0 buffer using a biomass:digestion buffer ratio of 1:2.5 (w/v).

Following digestion, the apoplastic fraction was filtered through a 400 μm nylon filter to remove coarse undigested vegetal tissue (<5% of starting biomass). The filtered extract was then centrifuged at room temperature for 15 min at 5000×g to remove protoplasts and intracellular contaminants (proteins, DNA, membranes, vesicles, pigments, etc). Next, the supernatant was depth-filtered (for clarification) using a 1.2 μm glass fiber filter (Sartorius Stedim) and a 0.45/0.2 μm filter (Sartorium Stedim), before being subjected to chromatography.

In order to prepare for chromatography, the extract may be concentrated by suitable methods known in the art, for example the extract may be centrifuge and the pellet resuspended in appropriate buffer and volume or the extract may be concentrated and diafiltrated on tangential flow filtration (TFF) systems, equipped with appropriate membrane prior to loading on chromatographic media. Membranes may be flat-sheet or hollow fibres, pore size may range between 100, 300, 500, 750 kDa or 1μ molecular weight cut-off Lumen size may be chosen according to supplier line of product, usually 0.75 to 1 mm lumen size is preferable. Shear rates may be adjusted between 2000 s⁻¹ to 10 000 s⁻¹ with appropriate retentate and permeate flow rates.

The clarified apoplastic fraction was loaded over a cation exchange column (Poros HS Applied Biosystems) equilibrated with an equilibration/elution buffer (50 mM NaPO₄, 100 mM NaCl, 0.005% Tween 80 pH 6.0). Once the UV was back to zero, the extract was step-eluted with the equilibration/elution buffer containing increasing concentrations of NaCl (500 mM). Where necessary, the chromatographic fractions were concentrated 10 times using Amicon™ devices equipped with 10 kDa MWCO. Protein analysis was performed as described in previous examples.

Finally, a last formulation approach may be employ to buffer exchange the final candidate vaccine, by methods known in the art. For example a centrifugation or a TFF step may be used for such purpose, as described above.

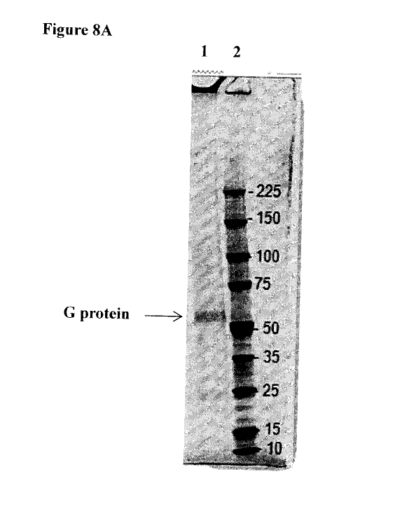

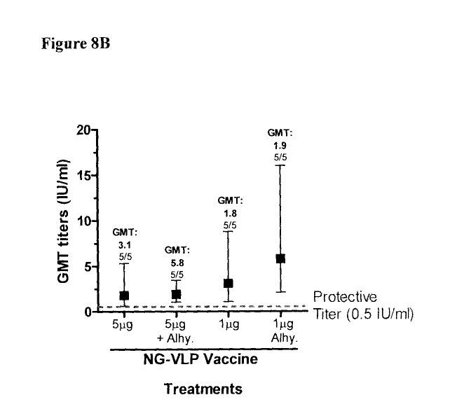

Under conditions described as above, purity of 75% or greater for Rab-VLP vaccines preparation with sufficient quality for immunogenicity study may be obtained. As illustrated in FIG. 8A, the G protein migrates to the expected molecular weight of ca. 55 kDa. The size of the Rab-VLP is estimated to be between 175-190 nm by dynamic light scattering (Zeta sizer 90, Malvern instrument), a particle size similar to that described for the Rabies virus.

### Immunization Studies in Mice

Immunogenicity of the Rab-VLP was evaluated in the Balb/c mouse model. Briefly, groups of female BALB/c mice (8-10 weeks old; Charles River Laboratories, USA) were vaccinated with a total of three injections (0.05 mL/site) on each immunization day (Study Days 0, 7 and 28) in the left and right hind limb musculature intramuscularly (i.m.) for a total of 0.1 ml volume injected that contained various doses of Rab-VLP vaccine (1 or 5 μg—with or without adjuvants). The doses were based upon G protein concentrations determined bybicinchoninic acid (BCA) assay combined with purity assessment by densitometry. Alhydrogel® at final concentration of 1% was used as the adjuvant. The placebo group was immunized by the same route and regimen as the candidate vaccine. Fifteen mice per group were used to provide adequate statistical power to the study. Serum samples were collected prior vaccination (pre-immune sera) and on day 7, 21 and 44. Five animals per group were sacrificed on day 7, 21 and 44 in order to perform rapid fluorescent focus inhibition test (RFFIT) to evaluate the protective antibody in the sera. The protective dose established by the World Health Organization (WHO) for Rabies vaccine is 0.5 international unit (IU) per ml. As shown in FIG. 8b, the non-adjuvanted Rab-VLP vaccine, with doses as low as 1 μg, allows for achieving higher titers than the standard titers established by the WHO.

All citations are hereby incorporated by reference.

The present invention has been described with regard to one or more embodiments. However, it will be apparent to persons skilled in the art that a number of variations and modifications can be made without departing from the scope of the invention as defined in the claims.

1-36. (canceled)

37. A method of producing a rabies virus like particle (VLP) in a plant comprising,
a) introducing a nucleic acid comprising a regulatory region active in the plant operatively linked to a nucleotide sequence encoding a native rabies glycoprotein (G), into the plant, or portion of the plant,
b) incubating the plant or portion of the plant under conditions that permit the expression of the nucleic acid, thereby producing the rabies VLP,
c) harvesting the plant, and
d) extracting the VLPs, wherein the VLPs range in size from 40-300 nm and,
wherein the VLP comprises a lipid obtained from a plasma membrane of the plant.

38. A method of producing a rabies virus like particle (VLP) in a plant comprising,
a) providing a plant or portion of the plant comprising a nucleic acid comprising a regulatory region active in the plant operatively linked to a nucleotide sequence encoding one or more native rabies glycoprotein (G),
b) incubating the plant or portion of the plant under conditions that permit the expression of the nucleic acid, thereby producing the rabies VLP,
c) harvesting the plant, and
d) extracting the VLPs, wherein the VLPs range in size from 40-300 nm, and
wherein the VLP comprises a lipid obtained from a plasma membrane of the plant.

39. The method of claim 37, wherein the nucleotide sequence further encodes, comprises or both encodes and comprises, one or more than one amplification element.

40. The method of claim 39, wherein the one or more than one amplification element is a geminivirus amplification element.

41. The method of claim 40, wherein the one or more than one geminivirus amplification element is selected from a Bean Yellow Dwarf Virus long intergenic region (BeYDV LIR), and a BeYDV short intergenic region (BeYDV SIR).

42. The method of claim 37, wherein a second nucleic acid comprising a second regulatory region active in the plant and operatively linked to a second nucleotide sequence encoding a rabies matrix protein (M) or fragment thereof is introduced into the plant, or portion of the plant and is expressed when incubating the plant or portion of the plant in step b).

43. The method of claim 38, wherein the plant or portion of the plant further comprises a second nucleic acid comprising a second regulatory region active in the plant and operatively linked to a second nucleotide sequence encoding a rabies matrix protein (M) or fragment thereof and is expressed when incubating the plant or portion of the plant in step b).

44. The method of claim 37, wherein in the step of introducing (step a), the nucleic acid is transiently expressed in the plant.

45. The method of claim 37, wherein, in the step of introducing (step a), the nucleic acid is stably expressed in the plant.

46. The method of claim 37, wherein the VLP is extracted by biochemical extraction.

47. A plant-derived VLP comprising one or more native rabies glycoprotein (G) wherein the VLP comprises a lipid obtained from the plasma membrane of the plant.

48. A VLP produced by the method of claim 37.

49. The VLP of claim 48, wherein the lipid comprises one or more than one lipid derived from the plant.

50. The VLP of claim 48, wherein one or more glycoprotein (G), comprises plant-specific N-glycans, or modified N-glycans.

51. A composition comprising an effective dose of the VLP of claim 48 for inducing an immune response in a subject, and a pharmaceutically acceptable carrier.

52. A method of inducing immunity to a rabies virus infection in a subject, comprising administering the composition of claim 51.

53. The method of claim 52, wherein the composition is administered to a subject orally, intradermally, intranasally, intramuscularly, intraperitoneally, intravenously, or subcutaneously.

54. The VLP of claim 48 for use in inducing immunity to a rabies virus infection in a subject.

55. A food supplement comprising the VLP of claim 48.

56. The method of claim 37, further comprising introducing an additional nucleic acid sequence, the additional nucleic acid sequence encoding a suppressor of silencing, a geminivirus replicase or both.

57. The method of claim 56, wherein the suppressor of silencing and the geminivirus replicase are encoded by two different additional nucleic acid sequences that are introduced into the plant, or portion of the plant.

58. The method of claim 56 wherein the suppressor of silencing is selected from the group HcPro and p19.

59. The method of claim 37, wherein the native rabies glycoprotein (G) has at least 90% sequence identity to SEQ ID NO: 14.

60. The method of claim 37, wherein the native rabies glycoprotein (G) has the amino acid sequence shown as in SEQ ID NO:14.

61. The method of claim 42, wherein the rabies matrix protein (M) has at least 90% sequence identity to SEQ ID NO: 6.

62. The method of 42, wherein the rabies matrix protein (M) has the amino acid sequence shown as in SEQ ID NO: 6.

63. The method of claim 37, wherein the nucleic acid sequence comprising the regulatory region is further operatively linked with one or more than one comovirus enhancer.

64. The method of claim 63, wherein the one or more than one comovirus enhancer is a comovirus UTR.

65. The method of claim 64, wherein the comovirus UTR is a Cowpea Mosaic Virus (CPMV) UTR.

66. The method of claim 37, wherein the VLP does not contain a viral matrix or a core protein.
